(19) United States
(12) Patent Application Publication
(10) Pub. No.: US 2012/0077253 A1
(43) Pub. Date: Mar. 29, 2012

(54) Methods and Systems for Controlled Illumination

(75) Inventors: James BURKHEAD, Memphis, TN (US); Thomas E. BROKAW, Reno, NV (US); Jason D. LICAMELE, Scottsdale, AZ (US); Aniket KALE, Chandler, AZ (US); Geoffrey MARGOLIS, Los Angeles, CA (US); Donald K. JURGENSEN, China Spring, TX (US); Shan QIN, Gilbert, AZ (US)

(73) Assignee: HELIAE DEVELOPMENT, LLC, Gilbert, AZ (US)

(21) Appl. No.: 13/194,675

(22) Filed: Jul. 29, 2011

Related U.S. Application Data
(60) Provisional application No. 61/369,380, filed on Jul. 30, 2010.

(51) Int. Cl. C12N 1/12

(52) U.S. Cl. 435/257.1

(57) ABSTRACT

Embodiments of the present invention relate generally to systems and methods for the collection of solar energy and the use thereof to generate an algal biomass capable of producing biofuels. In one implementation, the specific irradiance (e.g., moles photons per gram per day) is controlled to optimize algal growth and the production of polar lipids, non-polar lipids, and proteins. Optionally, non-photosynthetic portions of the light spectrum are diverted for uses other than illumination of the algae and/or are shifted in wavelength to fall within the photosynthetic spectrum

### CROSS REFERENCE TO RELATED APPLICATIONS

This application claims the benefit of U.S. Provisional Application No. 61/369,380, filed Jul. 30, 2010, the entire contents of which are hereby incorporated by reference herein.

### BACKGROUND

Algae have gained significant importance in recent years given their inherent advantage in solving several critical global issues such as the production of renewable fuels, reducing global climate change, wastewater treatment, and sustainability.

Algae's superiority as a biofuel feedstock arises from a number of factors such as high per-acre productivity when compared to typical terrestrial oil crop plants, non-food based feedstock resources, and its ability to be cultivated on otherwise non-productive, non-arable land.

Several thousand species of algae have been screened and studied for lipid production worldwide over the past several decades, of which about 300 rich in lipid production have been identified. The lipids produced by algae are similar in composition when compared to other contemporary oil sources such as oil seeds, cereals, and nuts.

As the United States has already consumed over 80% of its proven oil reserves, it currently imports more than 60% of its oil. It is anticipated that within 20 years the United States will be importing in the range of 80-90% of its oil. Much of this imported oil is supplied by nations in politically volatile regions of the world, a fact which poses a constant threat to a stable oil supply for the United States. Although the United States can continue to increasingly import foreign oil, global oil supplies are not infinite and importation continues to increase the United States trade deficit and create an increasing burden on the economy.

Commercial cultivation of lipid producing algae provides a solution to the growing problem of oil shortages and increases in cost of importation. Algae oil can be used to replace petroleum based products. Algae can be used to generate oil of varying lipid profiles for use in a variety of applications, including, but not limited to, the generation of diesel, gasoline, kerosene, and jet fuel.

### SUMMARY

Embodiments herein relate generally to systems and methods for culturing algae to produce fatty acids and hydrocarbons. In particular, embodiments described herein concern exposing algae cultures to a specific amount of light depending on the mass of cells present in the culture and keeping the light per gram of algae mass constant by increasing light intensity as the culture increases in density and/or removing algae mass over time to keep the amount of light per gram the same over the lipid producing period. This allows for maintenance of a constant rate of algal growth far beyond that achieved previously. This enables the production of large amounts of algal biomass, as well as algal oil and coproducts in greater quantities than achievable by conventional algal lighting methods.

There are a number of key process parameters that separately affect growth and oil production. These include, but are not limited to, light irradiance, carbon dioxide supply, pH, initial algal cell density, mass of algae being irradiated, and composition of the growth medium.

Embodiments of the invention include methods comprising providing an algal culture contained within a photobioreactor, the photobioreactor having at least one surface through which light may pass, exposing the at least one surface of the photobioreactor to a light source that emits photons such that at least a portion of the algal culture is exposed to a quantity of emitted photons over a given time period, periodically estimating the algal mass of the portion of the algal culture exposed to the quantity of emitted photons over the given time period, in response to measuring the algal mass, increasing or decreasing at least one of (i) the quantity of emitted photons to which the portion of the algal culture is exposed over the given time period or (ii) the algal mass exposed to said quantity of emitted photons over the given time period to maintain a ratio of the quantity of photons emitted over the given time period to the algal mass within a defined range.

In some embodiments of the invention, the algal culture is a batch algal culture or a continuous growth algal culture. In others, the light source is artificial light, natural light, or a combination of artificial and natural light. In yet other embodiments, the light source is artificial and emits photons that comprise mainly photosynthetically active radiation. In other embodiments, the defined range in which the ratio of the quantity of photons emitted over the given time period to the algal mass is maintained at less than or equal to a ratio at which a growth rate of the algal culture is maximal.

In still other embodiments, the rate at which the emitted photons are incident upon the surface of said photobioreactor is constant, and the defined range in which the ratio of the quantity of photons emitted over the given time period to the algal mass is maintained by removing at least a fraction of said algae mass as it grows over time. In further embodiments, the quantity of emitted photons to which the portion of the algae culture is exposed over time is increased in proportion to an increase in the algal mass due to algal culture growth to maintain the ratio of the quantity of photons emitted over the given time period to the algal mass. In yet further embodiments, the algal mass increase due to growth is at a rate of about 1 gram of algal mass per liter of algal culture per day.

Further embodiments also comprise applying an environmental stress to the algal culture to induce the algal culture to produce algal lipids. In some embodiments, the environmental stress is nitrate depletion, increased light, increased temperature, phosphate depletion, or nutrient depletion.

Some embodiments of the invention include a method comprising providing an algal culture contained within a first photobioreactor, the first photobioreactor having at least one surface through which light may pass, exposing the at least one surface of the first photobioreactor to a first light source that emits photons such that at least a first portion of the algal culture is exposed to a first quantity of emitted photons over a first time period, periodically estimating the algal mass of the first portion of the algal culture exposed to the first quantity of emitted photons over the first time period, in response to measuring the algal mass of the first portion, increasing or decreasing at least one of (i) the first quantity of emitted photons to which the first portion of the algal culture is exposed over the first time period or (ii) the algal mass of the first portion exposed to the first quantity of emitted photons over the first time period to maintain a first ratio of the first quantity of photons emitted over the first time period to the algal mass of the first portion within a first defined range, transferring a second portion of the algal culture contained within the first photobioreactor to a second photobioreactor, the second photobioreactor having at least one surface through which light may pass, applying an environmental stress to the second portion of the algal culture to induce the second portion of the algal culture to produce algal lipids, exposing the at least one surface of the second photobioreactor to a second light source that emits photons such that at least a third portion of the algal culture is exposed to a second quantity of emitted photons over a second time period, periodically estimating the algal mass of the third portion of the algal culture exposed to the second quantity of emitted photons over the second time period, in response to measuring the algal mass of the third portion, increasing or decreasing at least one of (i) the second quantity of emitted photons to which the third portion of the algal culture is exposed over the second time period or (ii) the algal mass of the third portion exposed to said second quantity of emitted photons over the second time period to maintain a ratio of the second quantity of photons emitted over the second time period to the algal mass of the third portion within a second defined range.

In further embodiments of the invention, at least one of the first and the second photobioreactor is a batch photobioreactor or a continuous growth photobioreactor. In still other embodiments, at least one of the first and the second light source is artificial light, natural light, or a combination of artificial and natural light. In yet others, at least one of the first and the second light sources is artificial and emits photons that comprise mainly photosynthetically active radiation.

In some embodiments, the first defined range in which the ratio of the first quantity of photons emitted over the first time period to the algal mass of the first portion is maintained less than or equal to a ratio at which a growth rate of the algal culture is sustainable. In others, the rate at which the emitted photons are incident upon the surface of the first photobioreactor is constant, and the first predetermined range in which the ratio of the first quantity of photons emitted over the first time period to the algal mass of the first portion is maintained by removing at least a portion of said algae mass of the first portion as it grows over time.

In still other embodiments, the first quantity of emitted photons to which the first portion of the algae culture is exposed over time is increased in proportion to an increase in the algal mass of the first portion due to algal culture growth to maintain the ratio of the first quantity of photons emitted over the first time period to the algal mass. In yet further embodiments, the first light source and the second light source are different. In even further embodiments, the environmental stress is nitrate depletion, increased light, increased temperature, phosphate depletion, or nutrient depletion. In some embodiments, the first defined range differs from the second defined range.

### BRIEF DESCRIPTION OF THE FIGURES

FIG. 1: Experimental setup including schematic for a panel photobioreactor.

FIG. 2: Plot of algae concentration over time as specific light irradiance was increased.

FIG. 3: Plot of algae growth rate in terms of grams per liter per day as specific light irradiance was increased.

FIG. 4: Plots of algae lipid content over time as specific light irradiance was increased from 0.6 (A) to 1 (B) to 1.2(C).

FIG. 5: Plot of algae lipid content in terms of percent increase per day as specific light irradiance was increased.

FIG. 6: Plot of the effect of illumination cycling on algae growth.

FIG. 7: Process flow diagram for a method to maintain specific light irradiation for optimum growth and oil production by batch oil conversion.

FIG. 8: Process flow diagram for a method to maintain specific light irradiation for optimum growth and oil production by continuous oil conversion.

FIG. 9: Cell density and productivity expressed in g/L-day for a batch culture of Nannochloropsis spp. cultured under a specific light value of 0.414 mol photons/g-day.

FIG. 10: Algae biomass concentration (g/L) plotted over time (days) for continuous cultures harvested at 25% (4 gram liter⁻¹) and 10% (8 g/L) under similar irradiance.

FIG. 11: Algae biomass concentration (g/L) plotted over time (days) in a continuous culture system with a 50% harvest rate and a specific light of 0.489 mol photons/g-day.

FIG. 12: Growth rate at selected specific light levels over time

FIG. 13: Plots of specific light oil phase biomass concentration (A); specific light oil phase lipid data (B); and specific light oil phase neutral lipid data (C).

FIG. 14: Extrapolated production curves based on continuous culture data.

FIG. 15: Predicted specific light curve for Nannochloropsis.

FIG. 16: PAR sunlight plot based on data collected in Phoenix Ariz., March 2011.

FIG. 17: Predicted algal culture concentration without specific light control.

FIG. 18: Predicted algal growth rates without specific light control.

FIG. 19: Schematic of an exemplary photobioreactor with specific light control.

FIG. 20: Predicted algal culture concentration with specific light control.

FIG. 21: Predicted algal growth rates with specific light control.

### DETAILED DESCRIPTION

The term “about” or “approximately” are defined as being close to as understood by one of ordinary skill in the art, and in one non-limiting embodiment the terms are defined to be within 10%, preferably within 5%, more preferably within 1%, and most preferably within 0.5%.

The terms “inhibiting” or “reducing” or any variation of these terms, when used in the claims and/or the specification includes any measurable decrease or complete inhibition to achieve a desired result.

The term “effective,” as that term is used in the specification and/or claims, means adequate to accomplish a desired, expected, or intended result.

The use of the word “a” or “an” when used in conjunction with the term “comprising” in the claims and/or the specification may mean “one,” but it is also consistent with the meaning of “one or more,” “at least one,” and “one or more than one.”

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

The terms “algae” and “algae strain” include both microalgae and cyanobacteria. In some embodiments, the algae are eukaryotic microalgae. In others, the algae are Nannochloropsis.

As used herein, a photobioreactor (PBR) is an industrial-scale culture vessel, which can be made of transparent and/or clear materials that can be successfully penetrated by photosynthetically active radiation (PAR) and enable algae to grow, proliferate, and/or produce oil. Nonlimiting examples of such materials include glass, acrylic, polycarbonate, plastics, and PVC.

For use in this aspect of the invention, any type of system or photobioreactor can be used, including, but not limited to, open raceways (e.g., slow ponds constructed as a loop, in which the culture is circulated by a paddle-wheel), closed systems, (e.g., photobioreactors made of transparent tubes or other transparent containers, in which the culture is mixed by either a pump or air bubbling), tubular photobioreactors, and flat plate-type photobioreactors.

As used herein, “photosynthetically active radiation” means the spectral range of light radiation from about 400 nm to about 700 nm wavelengths that photosynthetic organisms are able to use in the process of photosynthesis. This light radiation may be solar or may be emitted from an artificial light source, such as, but not limited to, LEDs, fluorescent bulbs, power compact bulbs, metal halide bulbs, and incandescent bulbs.

As used herein, “specific light” is the ratio of the moles photons per unit time irradiating a given algal mass divided by the mass of the algae being irradiated. As algal mass increases, the amount of moles photons per unit time increases accordingly to maintain the specific light within a defined range. Conversely, algal mass can be removed while the amount of moles photons per unit time is kept constant. In short, the key is to keep the ratio approximately constant, meaning operating within a defined range, by manipulating either the amount of moles photons per unit time or the algal mass. The surface area to volume ratio of the photobioreactor that the algae is grown in must be taken into account, and it is assumed that stirring of the algae within the reactor has been optimized.

Several operational ranges can be employed to practice the methods of the instant invention. In some embodiments, the defined operational range is between about 0.257 mol photons/g-day to about 0.499 mol photons/g-day. In others, the defined operational range is between about 0.4 mol photons/g-day to about 0.5 mol photons/g-day. In still others the defined operational range is between about 0.257 mol photons/g-day and about 0.98 mol photons/g-day. In yet others, the defined operational range is between about 0.5 mol photons/g-day and about 0.98 mol photons/g-day. In some embodiments, the defined range is about +/−50% of a selected specific light value. These values can be 0.414, 0.499, 0.6, and 0.76 mol photons/g-day.

Specific light is measured in units of moles photons per gram of algae per day and can be calculated for a given algal strain using the methods disclosed herein. While examples disclosed in the instant application are directed to Nannochloropsis, one of skill in the art, in light of this disclosure would be able to apply these methods and systems to any photosynthetic algal strain.

As used herein, “periodically” means at regular intervals, or at intervals of varying lengths. In essence, periodically means as needed or as determined is necessary by one of skill in the art in light of the conditions of the particular experiment, as determined required by the person practicing the invention of the instant disclosure.

As used herein “oil” and “algae oil” includes a composition produced by algae comprising one or more of fatty acids, hydrocarbons, glycolipids, phospholipids, triglycerides, neutral or polar lipids, and any other precursors that are generated by algae and can be used to generate fuel products or petroleum based product analogs such as, but not limited to, plastics.

Other objects, features and advantages of the present invention will become apparent from the following detailed description. It should be understood, however, that the detailed description and the examples, while indicating specific embodiments of the invention, are given by way of illustration only. Additionally, it is contemplated that changes and modifications within the spirit and scope of the invention will become apparent to those skilled in the art from this detailed description.

Current methods of algal biomass production face a number of obstacles. Currently algae are typically grown under constant light irradiation or under irradiation of the sun which varies throughout the course of the day. Under these conditions cultures are typically limited to continuous growth for only 5 to 6 days. While some cultures have been reported to survive longer, others have been unable to develop culture conditions that allow for optimal and sustained exponential growth. It has been discovered that controlling the ratio of light irradiation to algal mass can induce significant and unexpected increases in algal biomass production. This ratio can be manipulated by controlling either the amount of light irradiation or the mass of algae being irradiated.

Conventional methods do not entail the adjustment of algae culture cell density and/or changes in light irradiation intensity, as described in the instant application. Conventional methods generate cultures that operate at optimal growth levels for very short periods of time, unlike the methods disclosed herein, which allow for maintaining optimal and constant growth rates for far longer periods of time than previously known or achieved in the art.

When a conventionally grown algal culture is irradiated by sunlight, changing light conditions over the course of a day cause changes in light irradiation intensity on outdoor reactors and ponds. The intensity of sunlight can vary as much as 4 to 5 fold throughout the day. This means that the algal culture only achieves optimal growth for two relatively short periods during the course of the day. The rest of the time the culture is either getting too little light, causing slow growth, or too much light, resulting in algal cell death. This cycle of under and over-irradiation wastes available light energy as well as reducing algal biomass production and oil yields. As disclosed herein, irradiation of an algal culture with specific light results in ideal or near ideal rates of algal growth and/or oil production without waste of light energy.

Finally, conventional processes do not allow for sustaining continuous cultures over long periods of time. The methods and systems of the instant invention allow for continuous, long term growth of algal cultures, something that those in the field of algaculture have tried to achieve, but heretofore have not succeeded. Application of the methods and systems disclosed herein allow the maintenance of an algal culture at a constant rate of high growth. Thus far, the inventors have maintained an algal culture at a steady state of growth for 107 days, 114 days total.

Embodiments of the invention provide methods of and systems for optimal growing of an algae culture and producing algae oil with at least a portion of that culture. It has been discovered that by operating the algae growing process and algae oil production process each at a constant specific irradiance, within a defined range, as measured in moles photons per gram algae per day, algae growth and algae oil production yields can be substantially increased relative to known methods. Disclosed herein are unique process methodologies to control algae oil production such that the process operates at increased oil production yields.

It is believed that many conventional processes for algae oil production are cost prohibitive and cannot meet the production demand of the market. The disclosed processes are believed to have relatively lower operating costs due to the increased production of biomass and oil per unit of light, per acre of area dedicated to algae culture.

It is believed that by irradiating algae with light at a particular ratio of light power to algae mass, the algae growth, the production of oil, and the production of coproducts are maximized. This results in greater quantities of oil produced by the algae in order to make the production of algae fuels more practicable. Furthermore, the increase in coproduct quantities produced by the algae allows for the increased production of compounds for the formulation of foods and supplements.

It is proposed that there is an estimated theoretical maximum of oil that can be produced by algae, calculated as per K. M. Weyer, “Theoretical Maximum Algal Oil Production”, Bioenerg. Res., Oct. 8, 2009 (Weyer). The goal is to get as close as possible to this theoretical maximum by optimizing the conditions of algae culture, in particular, the degree of light irradiance.

While it was known in the art that algae grows in the presence of PAR, it was not known that algae growth rates and oil production rates could be increased and sustained at a high level by irradiating algae with a specific level of light irradiation relative to algae mass.

PAR can be provided by natural or artificial light sources, or a combination of the two. PAR can also be obtained by shifting non PAR wavelengths into the PAR region of the spectrum by a variety of known methods.

Surprisingly, it was found that when algae cultures were irradiated with about 0.49 moles photons per gram algae per day, as measured in light incident on the growth surface of the bioreactor, it was found that algae cell growth increased as compared with conventional methods of algaculture. Previously run experiments indicate the incident light does not penetrate more than about a centimeter past the surface of a photobioreactor, so all light measurements are done of the light incident on the surface of a photobioreactor.

This effect is achieved by holding the specific irradiance constant within a defined range throughout the desired growth stage of the culture. It was unexpected that light changes within such a narrow range would have such a dramatic impact on algae growth. Furthermore, it was found that when algae cultures were irradiated with about 0.98 range moles photons per gram algae per day, algae production of lipids increased substantially. It was noted that while oil content increases during the oil production phase, the mass of the algae remained roughly the same.

Different species of algae and mutants thereof can be grown under conditions disclosed herein to achieve optimal lipid profiles and then increased production of these lipids can be achieved through the use of methods that comprise the present invention. The proposed algae growth process results in greater yields as compared to algae grown outdoors or indoors by conventional processes. Furthermore, by culturing algae within the ideal specific light irradiation ranges, it is likely that algae death rates associated with high spikes in light irradiance seen with earlier designs will decrease. Finally, the methods and systems described herein are generic for any algae. However, it is understood that each particular algae strain may have unique ideal specific irradiation ranges. In view of the instant disclosure, one of skill in the art would be able to determine the ideal specific light irradiation ranges for a particular strain of photosynthetic algae.

One of the key control objectives that must be achieved is maintenance of a specific irradiation within a defined range during the algae culture. During the growth phase, the photobioreactor must be operated in a manner to consistently maintain the desired level of specific irradiation. A number of parameters can affect algal growth rates, even if specific lighting is applied to a culture. These parameters include, but are not limited to, temperature, pH, nutrient levels, nitrate levels, and carbon availability.

Interestingly, controlled manipulation of these factors can stress an algal culture such that oil production is induced. These stressors must be carefully tested and monitored to achieve the desired result as too much stress can inhibit oil production. In light of the instant disclosure, one of skill in the art would be able to develop methods allowing for controlled stressing of an algal culture in order to produce optimal levels of lipid accumulation. In preferred embodiments, these stressors include increases in specific light irradiation, nitrate depletion, and controlled increases in temperature.

Experiments done to examine the specific light effect and determine optimal ranges of irradiation were carried out. These experiments all indicate that as the amount of specific light irradiating a culture is increased from zero, an algae culture initially experiences an increase in growth rates. At a certain point, these growth rates reach a maximum, after which at some point growth rates drop off. This phenomenon can be exploited to determine the optimal amount of specific light irradiance to maximize algal growth rates.

Specific light irradiation can allow for continuous, high levels of growth of algal cultures. While it was originally thought that maximum algal growth rates, and therefore algal mass and oil production would be achieved by culturing algae at the peak of the specific light curve, it was unexpectedly discovered that operating at a specific light level that falls below that which gives the maximum algal growth rate for an algal strain yields higher, and importantly, sustainable production rates of algae and oil. It is believed that this may be because as the amount of specific light is dropped below the maximum, more light is available to the algae in the culture.

Furthermore, from a practical standpoint, the amount of light energy that can be applied to a given culture is limited due to the physical constraints of light available, both from natural and artificial light sources, as well as limitations on the amount of light irradiation that an algal culture can sustain before experiencing enough stress that growth rates and/or oil production are reduced. The methods of the instant invention allow for high production rates of algae mass and/or oil that are sustainable over long periods of time.

Operating at a specific light level that falls below that which gives the maximum algal growth rate results not only in cost savings in the case of irradiance with artificial light sources, but also protects the algae from being too close to the point of over-irradiation. Too much light results in algal bleaching and cell death. High levels of light may also raise the temperature of the culture to the point of reducing cell growth and oil production rates.

Preferred embodiments of methods to accomplish this goal include, but are not limited to, a continuous operating mode which maintains the algae concentration constant and therefore the light source can remain constant, or a batch operating mode where the photon supply must be increased so as to maintain the moles photons per gram algae per day constant as the algae culture increases in density over time. In yet other embodiments, algal flow rates are manipulated to control specific light within a narrow range. In some preferred embodiments, the culture is diluted to maintain the ratio of moles photons per gram algae per day. In other preferred embodiments, the amount of illumination is increased as the density of the algae culture increases to hold the ratio of moles photons per gram algae per day constant within a defined range. In still other preferred embodiments, algae mass is removed from the culture in order to maintain the ratio of moles photons per gram algae per day. In yet other preferred embodiments, the specific amount of light is provided by either direct or indirect sunlight. In further preferred embodiments, the specific amount of light is provided for by artificial lights and/or a combination of sunlight and artificial light.

During the oil conversion phase of the process, the percentage of oil within the algae cells increases over time without a significant increase in the dry weight of the algae. Furthermore, this portion of the process is less sensitive to a slight shift in the moles photons per gram algae per day ratio. Thus, ratio values within a range will maintain oil production at its maximum, maintaining the mass of the algae culture and the level of specific irradiation the same throughout the process is believed to be sufficient to achieve the goal of maximal oil production.

The systems and methods disclosed herein comprise, but are not limited to, providing a first quantity of algae cells; determining a mass of the first quantity of algae cells; determining a selected quantity of light power to which the first quantity algae cells are to be exposed based on the mass of the first quantity of algae cells; and exposing the first quantity of algae cells to the selected quantity of light power, thereby producing a second quantity of algae cells.

### DESCRIPTION OF EXEMPLARY EMBODIMENTS

### Example 1

### Algae Growth at Different Specific Irradiation Levels

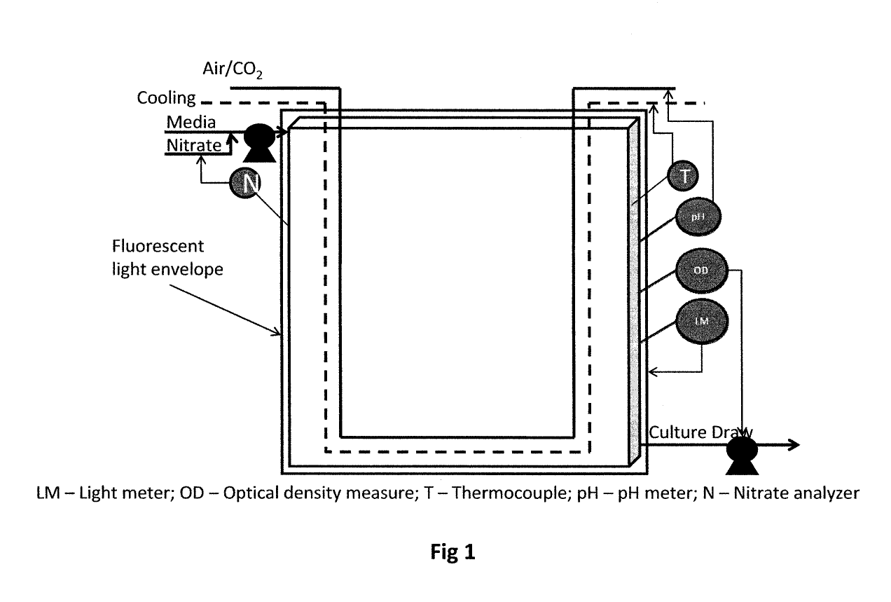

Nannochloropsis sp. algae were cultured in a 4′×4′×1.5″ panel photobioreactor with a 50 L volume at a starting concentration of 0.7 g/L. FIG. 1 is a schematic of the equipment used to conduct this set of experiments. The equipment also included a light meter, an optical density meter, a thermocouple, a pH meter, and a nitrate analyzer. This experiment examined the effect of specific light irradiance on the algal growth rate in a batch culture.

The photobioreactor was cooled using galvanized steel piping and a cold water reservoir. Sparging was used for increased mixing with compressed air. CO₂ was added to the air line and varied between 1-10% (v/v) of the air flow which was 0.5-2 vvm. Samples were taken every three hours between 8:00 AM and 5:00 PM each day and tested for temperature, pH, dry weight, and oil content. The pH was maintained at around 7.9 and the temperature at around 30° C. or less.

The culture was irradiated using artificial light. The number of light bulbs was changed throughout the experiment to control the level of specific light irradiance in order to demonstrate that maintaining a culture under an approximately constant level of specific light irradiation results in an approximately constant level of algae growth, as well as oil production. The culture was irradiated (24 hours per day of continuous lighting) with the light output from 2 bulbs for 2 days, then 6 bulbs for 7 days, and finally with 16 bulbs for one day. Equal numbers of 51 Watt blue (approximately 400-500 nm wavelength) and red (approximately 600-700 nm) fluorescent light bulbs were used throughout the test.

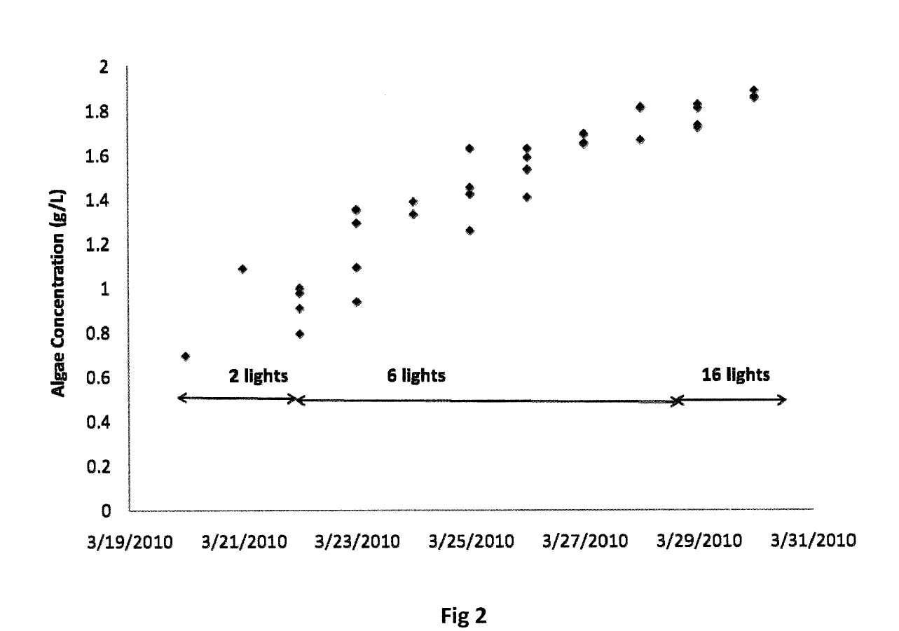

Because the algae growth was continuous and light levels were increased in a step-wise manner, specific light was not perfectly controlled and varied somewhat throughout the course of the experiment. Several samples were taken at each time point and the dry weights of the algae in each sample determined. These values were plotted and curves were fit to the points using known methods. FIG. 2 presents the algae concentration as a function of time and the number of bulbs used to irradiate the algae culture. From this data, it was demonstrated that increasing light levels as algal mass increased resulted in the growth rate of the algae remaining constant, rather than leveling off as is typically seen in algaculture utilizing a fixed amount of light, rather than the approximately fixed specific light ratio employed in the instant experiment.

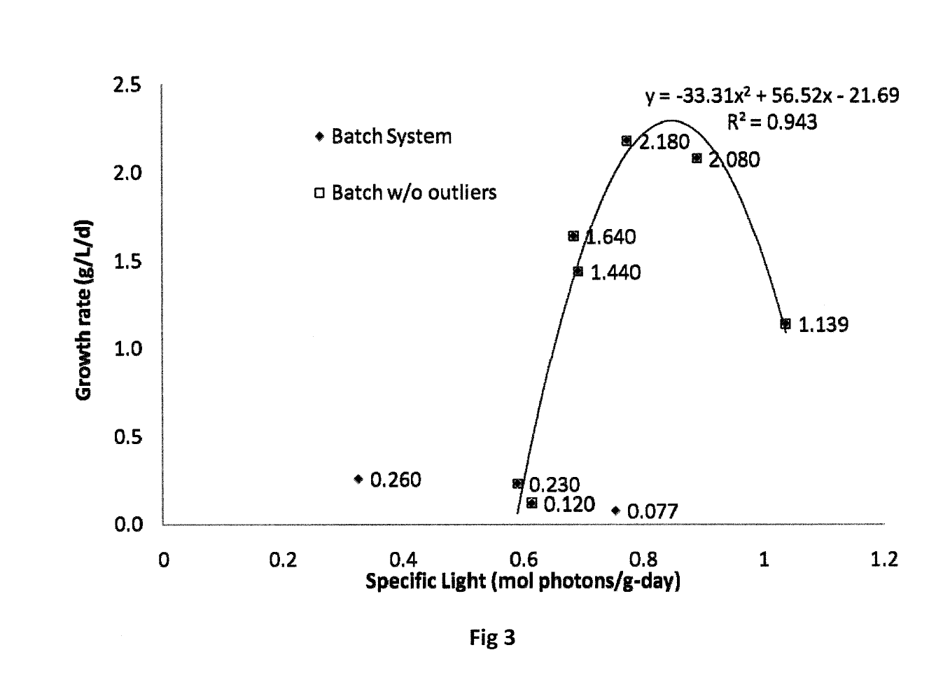

Algae growth rate is equal to the slope of a line plotting algae dry weight concentration against operating time. The growth rate of algae during a given interval can be calculated based on the change in algae concentration between two consecutive readings, divided by the length of the interval between the readings. The specific light irradiance was calculated by multiplying the measured incident light irradiation using a StellarNet Spectrometer (StellarNet Inc., Tampa Fla.) by the area of the PBR surface being irradiated and dividing by the average weight of algae in the PBR during the period of light measurement. It was later discovered that in this experiment, the StellarNet Spectrometer was improperly calibrated. The measured values were then corrected by determining the amount of light emitted by each bulb, as measured by a LI-COR® 250A PAR Lightmeter (LI-COR® Biosciences Lincoln, Nebr.) and then adjusting the values according to the number of bulbs used at each stage of the experiment. FIG. 3 is a plot of algae growth rates versus these corrected specific light values.

FIG. 3 presents the growth rate in grams per liter per day as a function of specific irradiance. Note that because of the spectrum of artificial lights used, as described above, the light energy portion of the specific irradiance value is essentially pure PAR light energy. The values plotted in the graph were calculated using this assumption. As the experiment was uncontrolled, and as previously mentioned, specific light was not strictly regulated, some of the data falls outside of the curve shown in FIG. 3. While there is no statistical basis for removing the data, removal of the outlying points, as marked in FIG. 3, results in a curve with 94.3% accuracy. Removal or manipulation of the other points on the curve leads to large reduction in the accuracy of the curve plot, indicating that this model has a significant amount of leverage. As a result, this experiment may be best utilized as a screening experiment, demonstrating the specific light phenomenon, and used to determine the direction of future experimentation.

From FIG. 3, it was concluded that algae growth rates have a maximum at a certain level of specific irradiation. The data gathered indicate that algae demonstrate the highest levels of growth when irradiated in the range of about 0.2-1 moles photons per gram algae per day. A preferred level of specific irradiation for maximum algae growth occurs at about 0.8 moles photons per gram algae per day. It is thought that irradiating algae during the growth phase of culture at a constant specific irradiation level within the range of 0.8 moles photons per gram algae per day results in maintenance of a constant high rate of algal growth. Since the data are for a batch system and relatively instantaneous growth rates and specific light conditions, this figure primary serves as a proof of the specific light concept and that specific light increases cause growth rates to go through a maximum. The data obtained supports the hypothesis that applying the maximum level of specific irradiation should lead to high levels of sustainable algae growth rates.

### Example 2

### Algae Oil Production at Different Specific Irradiation Levels

Algae were cultured in a 4′×4′×1.5″ panel photobioreactor with a 50 L volume at a starting concentration of 1.6 g/L. The photobioreactor was cooled using galvanized steel piping and a cold water reservoir. Sparging was used for increased mixing with compressed air. CO₂ was added to the air line and varied between 1-10% (v/v) of the air flow which was 0.5-2 vvm. Samples were taken every three hours between 8:00 AM and 5:00 PM each day and tested for temperature, pH, dry weight, and oil content. The pH was maintained at around 7.9 and the temperature at around 30° C. or less. FIG. 1 is a schematic of the equipment used to conduct this set of experiments. This experiment examined the effect of specific light irradiance on the oil production rate of algae in a batch culture. The culture was irradiated for 24 hours per day for the duration of the experiment at the light intensities described below.

Once the growth phase of the experiment was complete, as described in Example 1, supplementation of the culture with nitrate was stopped, thereby leading to nitrate depletion. This physiological stress induced the algae to cease growing and begin production of oil. The nitrate depleted culture was irradiated using artificial light in the form of the 16 bulb arrangement described above in connection with the growth phase.

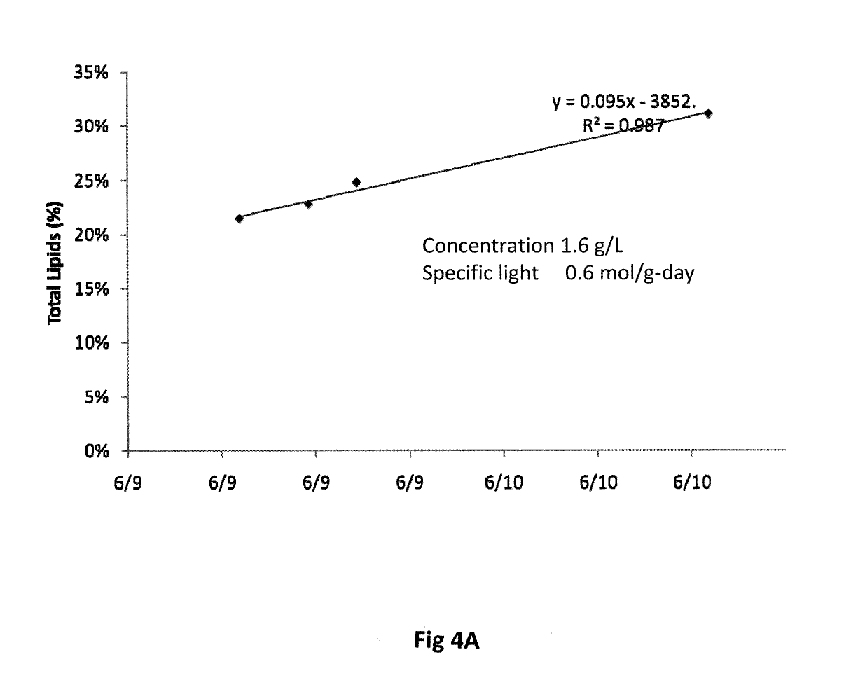

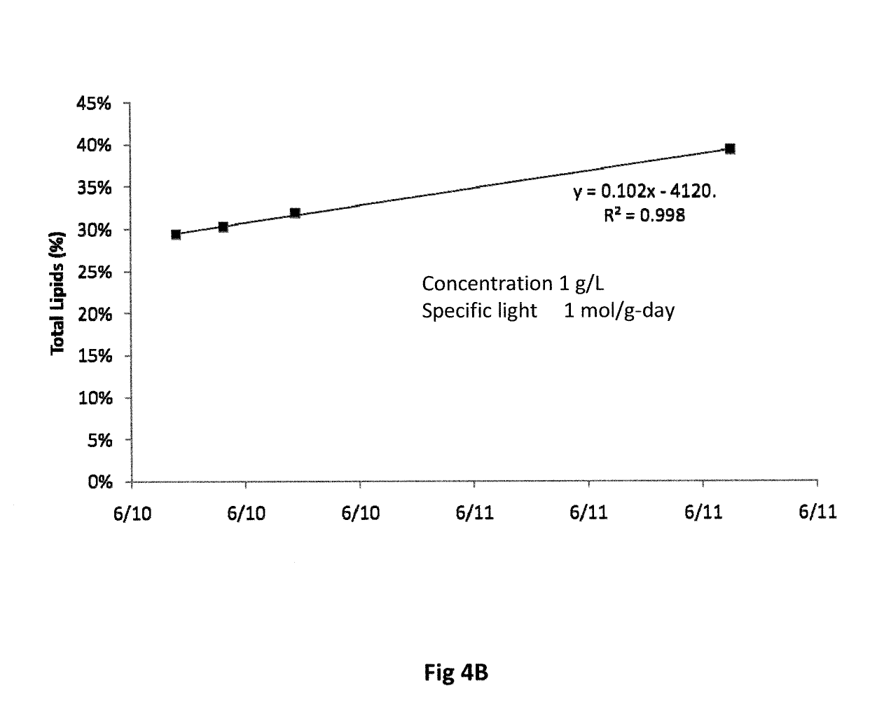

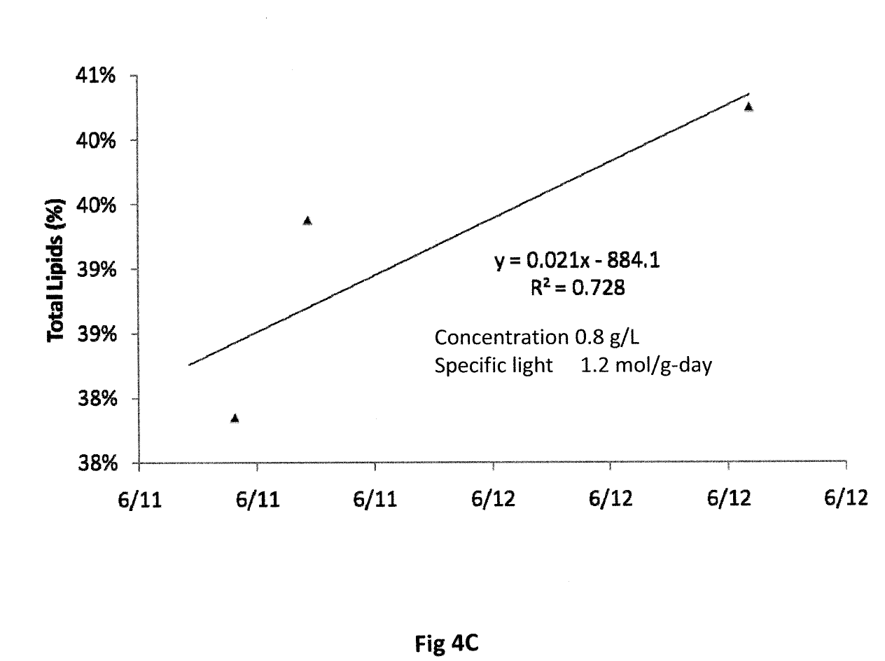

As the starting culture had a cell density of 1.6 g/L, the use of 16 bulbs resulted in the culture being irradiated at a level of about 0.6 moles photons per gram algae per day. Algae lipid content went from 21% to 31% in 24 hours, as seen in FIG. 4A. In order to obtain 1.0 moles photons per gram algae per day of irradiation, the algae culture was diluted to 1 g/L and irradiation with the 16 bulbs continued. Algae lipid content increased from 29% to 39% in 24 hours, as seen in FIG. 4B. Finally, due to limitations of the light fixture used (maximum of 16 bulbs), in order to obtain 1.2 moles photons per gram algae per day of irradiation, the culture was diluted to 0.8 g/L and irradiated with the 16 bulbs to achieve the desired specific light irradiation. Algae lipid content increased from 38% to 41% in 24 hours, as seen in FIG. 4C.

The oil conversion rate was calculated as being equal to the slope of the curve to obtain the oil conversion. In other words, the percentage change in oil content during the 24 hour period is the change in oil percent per day. The specific light irradiance was calculated by multiplying the measured incident light irradiation using StellarNet Spectrometer by the area of the PBR surface being irradiated and dividing by the average weight of algae in the PBR during the period of light measurement. It was later discovered that in this experiment, the StellarNet Spectrometer was improperly calibrated. The measured values were then corrected by determining the amount of light emitted by each bulb, as measured by a LI-COR® 250A PAR Lightmeter (LI-COR® Biosciences Lincoln, Nebr.) and then adjusting the values according to the number of bulbs used at each stage of the experiment.

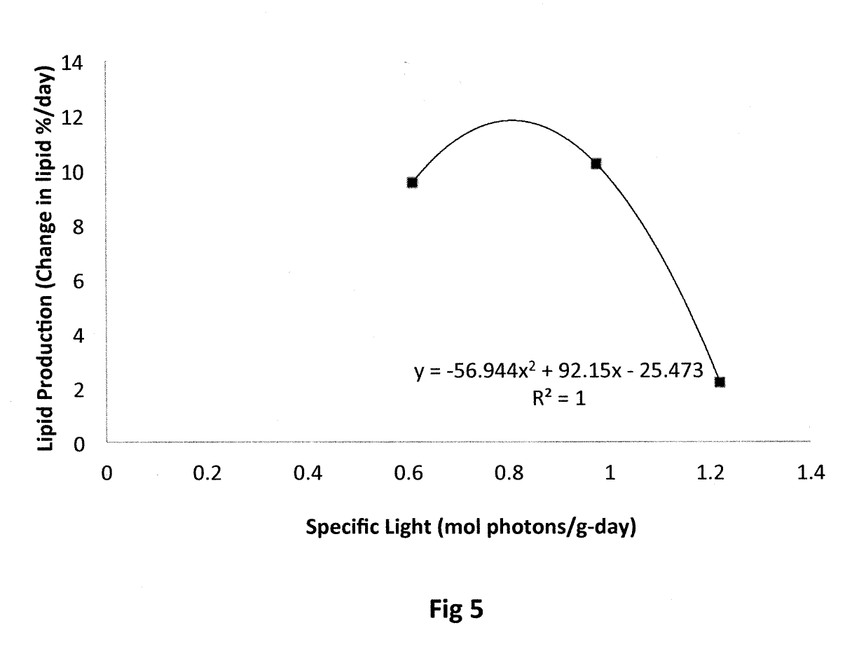

As mentioned above, because the algal mass remained roughly the same throughout the oil phase, specific irradiance levels remained constant throughout the course of the oil phase of the instant experiment. FIGS. 4A, 4B, and 4C present the algae oil content as a function of time. FIG. 5 presents the approximate oil production rate expressed in oil percent increase per day as a function of the specific irradiation in moles photon per gram algae per day. Similar to what was seen the growth phase experiment described in Example 1, oil production rates also appear to have a maximum at a certain specific light. As this experiment lacked a control, and the originally collected data was found to be inaccurate in certain respects, this data was not used to make any predictions, but rather to develop further experiments as described below.

A preferred level of specific irradiation for the maximization of algae oil production rates occurs at about 1 moles photons per gram algae per day. It is thought that irradiating algae during the oil production phase of culture at a constant specific irradiation level within the range of about 0.6-1.2 moles photons per gram algae per day results in increased algae oil production rate. The data indicates that under- or over-irradiating an algae culture results in lower rates of oil production as compared to algae cultured under specific light conditions.

### Example 3

### Effect of Illumination Cycles on Algae Growth

Using the testing apparatus and culture conditions described above, illumination cycles were tested. In Examples 1 and 2, the culture were continuously irradiated for 24 hours per day due to the belief that algae growth could be maintained at high production levels even when continuously irradiating algae for periods longer than 12 hours at a time. Conventional algaculture typically utilizes a day and night cycle to allow algae time for night respiration.

Using an automated light switching timer, the lights were turned off for 5 hours each night from 1:00 AM to 6:00 AM, providing a light-dark cycle condition (herein 3.8:1 light-dark cycle) at about 0.57 moles photons per gram algae per day. Another batch of algae was irradiated by having the light cycle on and off every 15 minutes (herein 1:1 light-dark cycle).

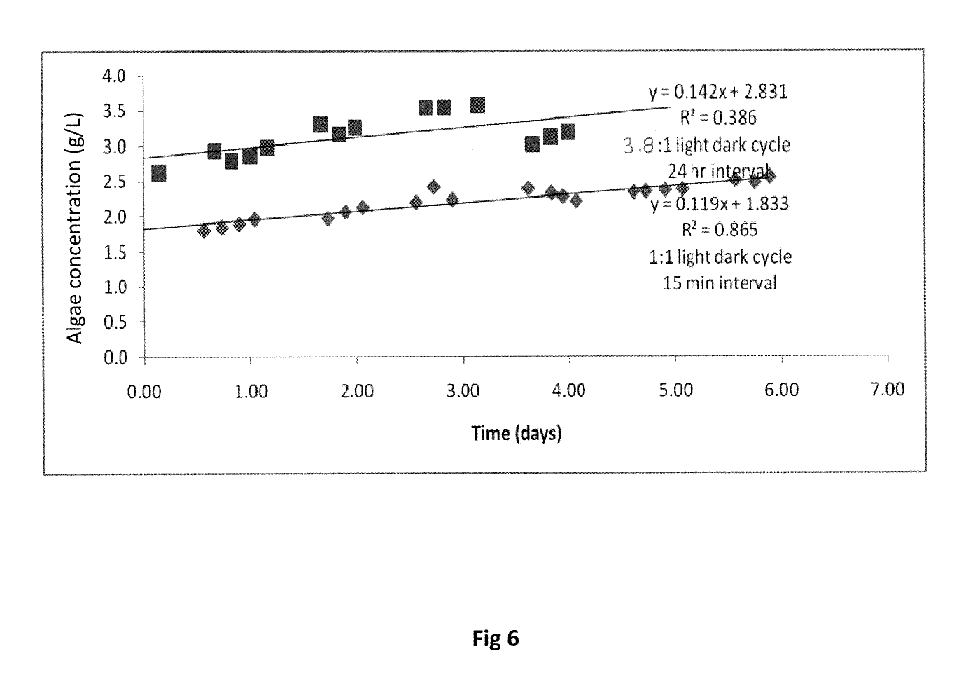

The data was plotted and curves were fit to the points using known methods. As shown in FIG. 6, based on the slopes of the growth curves, the growth rate of algae exposed to the 1:1 light-dark cycle was 0.11 g/L-day, which is less than that of the algae exposed to the 3.8:1 light-dark cycle which was 0.142 g/L-day. Note that the algae exposed to the 3.8:1 light-dark cycle had a lower growth rate than that generally observed for a 24 hour light operation, as seen in FIG. 3 where the growth rate was approximately 0.23 gram per liter per day under the same specific lighting conditions. Based on the results shown in FIG. 6, as compared to FIG. 2 and FIG. 3, it was concluded that Nannochloropsis sp. can grow continuously for at least 4-6 days without requiring time without light irradiation for night respiration. This conclusion is supported by the continued growth of the algae during the two different illumination cycles.

### Example 4

Continuous Growth With Batch Oil Production for Increased Growth and Oil Production Rates

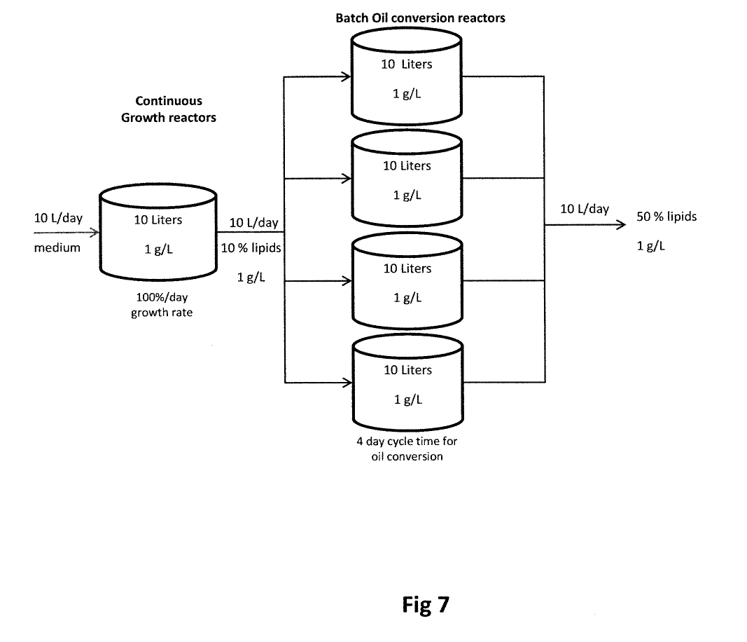

One method of maintaining specific irradiance at the appropriate levels through the growth phase and oil production phase involves splitting up the algae at the end of the growth phase into several PBRs for the purposes of carrying out the oil production phase. An exemplary schematic for this process appears in FIG. 7.

The process parameters for the growth phase will involve exposing an algae culture in a 4′×4′×1.5″ thick photobioreactor seeded with an initial algae cell density of 1-2 g/L with about 0.04-1 moles photons per gram algae per day and supplementing the growth medium with CO₂, macronutrients, including but not limited to nitrates (100-300 ppm) and phosphates (5-10 ppm), micronutrients such as trace metals, maintaining the pH between 6.5 and 8.5, maintaining the temperature between 25° C. and 27° C., and agitating the culture by the use of gas injection at a rate of 0.5-2 vvm as well as mechanical agitation. The average residence time of the algae in the continuous growth reactor is 24 hours. Note that while some algal cells have a residence time that is far shorter or far longer than 24 hours, on average the residence time is 24 hours.

The process parameters for the oil phase will involve irradiating the growth culture after nitrate depletion with about 0.1-1 moles photons per gram algae per day, maintaining the pH between 6.5 and 8.5 and maintaining the temperature between 27° C.-32° C., while continuing the gas and mechanical mixing used in the growth phase. The continuous growth reactor depicted in FIG. 7 is being continuously fed with medium 24 hours a day while the same amount of algal culture is being removed. In the instant example, 10 L of volume is added at a predesignated flow rate while the same amount of algal culture is simultaneously flowing into one of four batch oil conversion reactors, as described below.

The algae from the continuous growth reactor, operating at an algae mass density of 1 g/L, is separately fed daily into one of a series of four batch oil conversion reactors in four day cycles. On the first day, the first batch oil conversion reactor is filled with 10 L of algal culture and allowed to remain in the reactor. On the second day, the second batch oil conversion reactor is filled in the same manner as the first. On the third day, the third batch oil conversion reactor is filled in the same manner as the second. On the fourth day, the fourth batch oil conversion reactor is filled in the same manner as the third. At the end of the fourth day, the first batch oil reactor is emptied so that it is ready to repeat the cycle on the fifth day, i.e., the first day of the next four day cycle. This is repeated on each consecutive day with each consecutive reactor. This system allows for the algae to remain in a batch oil phase reactor for four days and then harvested for oil.

### Example 5

### Continuous Growth With Continuous Oil Production for Optimum Growth and Oil Production Rates

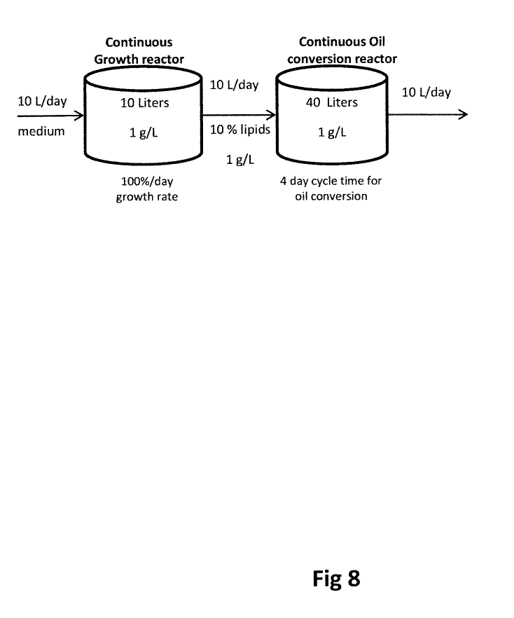

A second method of maintaining specific irradiance at the appropriate levels through the growth phase and oil production phase involves feeding the algae from the continuous growth reactor operating at an algal biomass density of about 1 g/L into a larger continuous oil conversion reactor for the purposes of carrying out the oil phase. An exemplary schematic for this process appears in FIG. 8.

The process parameters for the growth phase will involve exposing an algae culture in a 4′×4′×1.5″ thick photobioreactor seeded with an initial algae cell density of 1-2 g/L with about 0.04-0.4 moles photons per gram algae per day and supplementing the growth medium with CO₂, macronutrients, including but not limited to nitrates (100-300 ppm) and phosphates (5-10 ppm), micronutrients such as trace metals, maintaining the pH between 6.5 and 8.5, maintaining the temperature between 25° C. and 27° C., and agitating the culture by the use of gas injection at a rate of 0.5-2 vvm as well as mechanical agitation. In the instant example, the average residence time of algae in the continuous growth reactor is 1 day while the average residence time of the algae in the continuous oil conversion reactor is 4 days. Note that while some algal cells have a residence time that is far shorter or far longer, these given values are the average residence time.

The process parameters for the oil phase will involve irradiating the growth culture after nitrate depletion with about 0.1-1 moles photons per gram algae per day, maintaining the pH between 6.5 and 8.5, maintaining the temperature between 27° C.-32° C., while continuing the gas and mechanical mixing used in the growth phase.

Nutrient starvation is also a factor contributing to oil production during the oil phase. In particular, the depletion of nitrate can be used to induce oil production. When nitrates are no longer added to the medium, algae will eventually use it up, thereby leading to nitrogen depletion. It has been noted that when this occurs, algal biomass remains approximately constant while lipid synthesis increases.

In the instant example, the algae from the continuous growth reactor is fed into a continuous oil reactor at a rate of 10 L per day. The continuous growth reactor is simultaneously fed a supply of culture medium at the same rate, thereby maintaining the 10 L culture volume. The continuous oil reactor in the instant example has a volume of 40 L. The algae culture from the continuous growth reactor is fed into the continuous oil conversion reactor at a rate of 10 L per day. Since the volume of the continuous oil conversion reactor is 4 times that of the volume of culture entering/leaving the continuous oil conversion reactor, the average residence time of the algae is 4 days in the oil production phase.

### Example 6

### Control of Specific Light Irradiation

Control of specific irradiation levels can be achieved through a variety of ways. When sunlight is utilized as a light source, baffles, light filters, prisms, mirrors, and other methods for control of light intensity and spectra can be used to reduce the amount of light provided to the algae culture. A preferred embodiment comprises the culture of algae, suspended in a fluid medium, in a receptacle. The algae are then pumped through transparent tubing through which PAR is capable of penetrating. The culture is irradiated as it passes through the tubing in order to provide the algae with the light necessary for either growth or oil production, depending on the stage of the process. As the algae proliferate and culture density increases, the flow rate of the algae through the irradiated tubing can be adjusted such that specific irradiance levels are maintained and the same number of photons is provided per gram of algae. Flow rate can also be adjusted in response to changing light levels throughout to day or to compensate for change in weather, such as cloudiness, that impact the quantity of sunlight.

These methods also can be used in conjunction with artificial light, should this be needed. When artificial light is used, specific irradiance can also be controlled by changing the number of bulbs, the wattage of bulb, or through the use of dimmers. These examples are not limiting and one of skill in the art would understand that there are many ways to control the duration, intensity, and spectrum of both natural and artificial light.

### Example 7

### Residence Time of Algae in Cultures

A method of reducing inconsistency in the algae culture during the oil phase will involve moving the cultures through a series of batch reactors in order to reduce the residence time distribution of any algae in any particular reactor. In a well-mixed, continuously stirred reactor, as some culture is removed periodically in order to maintain an appropriate specific irradiance, it is possible that some of the algae removed will be significantly older or younger than the age required by the process. This is because as algae is removed, and medium is added, newer algae may flow out of the reactor in less than one day. Conversely, some algae may remain in the reactor for longer than one day. Over time, if the reactor is operated continuously, it will become filled with algae that have been in the culture for various periods, which is referred to as the residence time distribution.

To minimize the effects that overly long or overly short residence times might have on oil production, moving the algae to different reactors in series will reduce the residence time distribution, and, thus, would reduce any effects of increased or decreased residence time by ensuring the uniformity of the cultures. Similarly, a set of batch reactors can be fed sequentially by a feed reactor to insure a minimum residence time for all algae in a particular batch reactor.

In one exemplary embodiment, the algae are maintained in the growth phase for one day and then in the oil phase for four days. During the oil phase, the algae will flow through a set of reactors in sequence. This will reduce the residence time distribution of the culture.

### Example 8

### Demonstrating that Algae Will Grow at a Constant Growth Rate When Irradiated with Constant Specific Light Radiation within a Defined Range

Traditionally when algae are grown in a batch PBR they are irradiated with a constant light source. Under these light conditions, the algae start growing after a so-called lag phase, then grow rapidly at an increasing and accelerating growth rate for a period of time (exponential phase) before finally completely stopping growth.

On the other hand when algae are grown under conditions of constant specific light within a defined range, it is possible to maintain their growth rates at a constant and elevated level for long periods of time.

As algae grow in a batch PBR, their concentration in the reactor increases. By adjusting the number of light bulbs irradiating the PBR and the depth of the PBR utilized (which affects the total algae volume being irradiated) it is possible to maintain the specific light constant as the algae concentration changes. For any given algae concentration, Table 1 indicates the number of light bulbs and required PBR depth sizes required to maintain the specific light constant at 0.414 mol photons/g-day.

A series of batch PBR experiments were undertaken in flat panel PBRs of similar length (2 ft) and width (2 ft) but different depths (which is the distance between the two flat panel sides). The various PBR's utilized had depths of 2 inches, 1 inch, 0.5 inches and 0.25 inches.

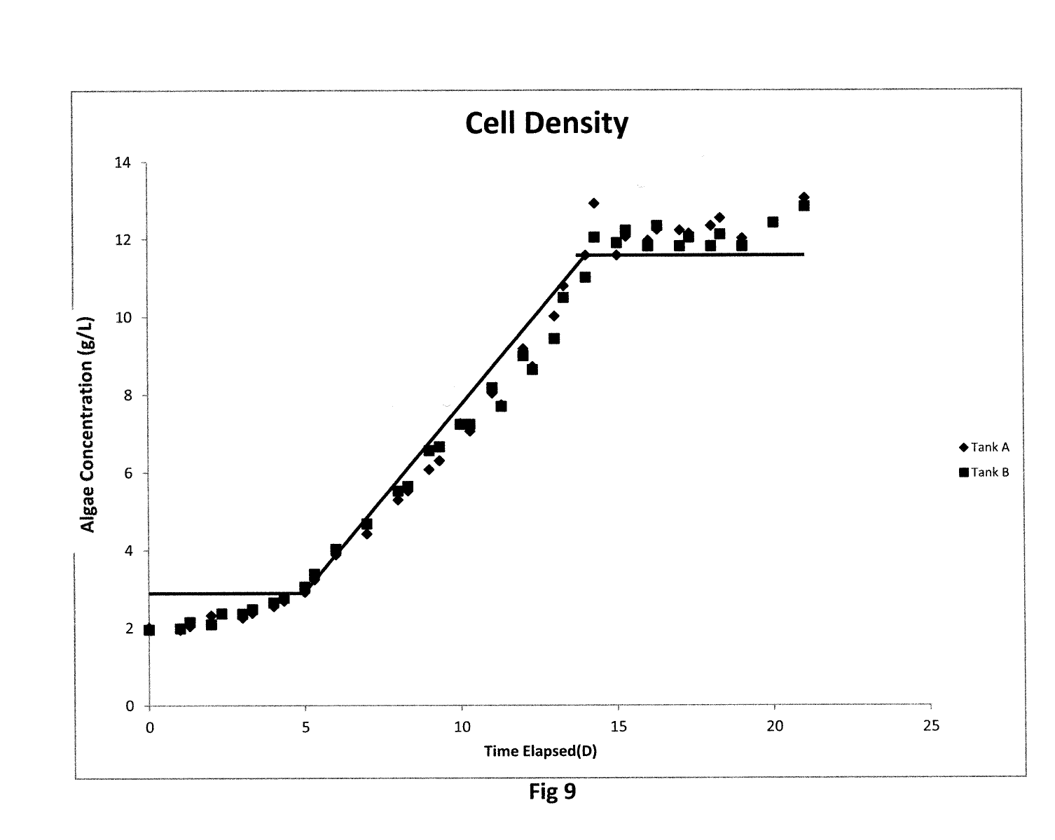

The conditions of Table 1 were used to maintain the specific light constant at 0.414 mol photons/g-day and the data of various experiments are shown in FIG. 9.

Clearly, after an initiation period, the Nannochloropsis sp. algae grew at a constant rate of 0.96 g/L-day over a 10 day period. The algae stopped growing rather abruptly as the concentration reached about 11 g/l. It is hypothesized that this may be due to the fact that algae concentration become so dense that it became difficult for light to penetrate any distance into the biomass.

TABLE 1

| Concentration | Number | Flux per Bulb | Flux | Pathlength | Distance from | Specific Light |
|---|---|---|---|---|---|---|
| (g/L) | of Bulbs | (umol/m\hat{ }2-s) | (umol/m\hat{ }2-s) | (in) | Bulb (in) | (mol/g-day) |
| 0.42 | 1 | 71.14 | 102.69 | 2 | 2.25 | 0.41 |
| 0.71 | 2 | 71.14 | 173.83 | 2 | 2.25 | 0.41 |
| 1.01 | 3 | 71.14 | 244.97 | 2 | 2.25 | 0.41 |
| 1.30 | 4 | 71.14 | 316.11 | 2 | 2.25 | 0.41 |
| 1.59 | 5 | 71.14 | 387.25 | 2 | 2.25 | 0.41 |
| 1.88 | 6 | 71.14 | 458.39 | 2 | 2.25 | 0.41 |
| 2.18 | 7 | 71.14 | 529.53 | 2 | 2.25 | 0.41 |
| 2.47 | 8 | 71.14 | 600.67 | 2 | 2.25 | 0.41 |
| 2.76 | 9 | 71.14 | 671.81 | 2 | 2.25 | 0.41 |
| 3.05 | 10 | 71.14 | 742.95 | 2 | 2.25 | 0.41 |
| 3.34 | 11 | 71.14 | 814.09 | 2 | 2.25 | 0.41 |
| 3.64 | 12 | 71.14 | 885.23 | 2 | 2.25 | 0.41 |
| 3.93 | 13 | 71.14 | 956.37 | 2 | 2.25 | 0.41 |
| 4.22 | 14 | 71.14 | 1027.51 | 2 | 2.25 | 0.41 |
| 4.51 | 15 | 71.14 | 1098.65 | 2 | 2.25 | 0.41 |
| 4.81 | 16 | 71.14 | 1169.79 | 2 | 2.25 | 0.41 |
| 4.94 | 8 | 71.14 | 600.67 | 1 | 2.25 | 0.414 |
| 5.52 | 9 | 71.14 | 671.81 | 1 | 2.25 | 0.414 |
| 6.10 | 10 | 71.14 | 742.95 | 1 | 2.25 | 0.414 |
| 6.69 | 11 | 71.14 | 814.09 | 1 | 2.25 | 0.414 |
| 7.27 | 12 | 71.14 | 885.23 | 1 | 2.25 | 0.414 |
| 7.86 | 13 | 71.14 | 956.37 | 1 | 2.25 | 0.414 |
| 8.44 | 14 | 71.14 | 1027.51 | 1 | 2.25 | 0.414 |
| 9.03 | 15 | 71.14 | 1098.65 | 1 | 2.25 | 0.414 |
| 9.61 | 16 | 71.14 | 1169.79 | 1 | 2.25 | 0.414 |
| 9.87 | 8 | 71.14 | 600.67 | 0.5 | 2.25 | 0.414 |
| 11.04 | 9 | 71.14 | 671.81 | 0.5 | 2.25 | 0.414 |
| 12.21 | 10 | 71.14 | 742.95 | 0.5 | 2.25 | 0.414 |
| 13.38 | 11 | 71.14 | 814.09 | 0.5 | 2.25 | 0.414 |
| 14.55 | 12 | 71.14 | 885.23 | 0.5 | 2.25 | 0.414 |
| 15.72 | 13 | 71.14 | 956.37 | 0.5 | 2.25 | 0.414 |
| 16.88 | 14 | 71.14 | 1027.51 | 0.5 | 2.25 | 0.414 |
| 18.05 | 15 | 71.14 | 1098.65 | 0.5 | 2.25 | 0.414 |
| 19.22 | 16 | 71.14 | 1169.79 | 0.5 | 2.25 | 0.414 |
| 19.74 | 8 | 71.14 | 600.67 | 0.25 | 2.25 | 0.414 |
| 22.08 | 9 | 71.14 | 671.81 | 0.25 | 2.25 | 0.414 |
| 24.42 | 10 | 71.14 | 742.95 | 0.25 | 2.25 | 0.414 |
| 26.76 | 11 | 71.14 | 814.09 | 0.25 | 2.25 | 0.414 |
| 29.09 | 12 | 71.14 | 885.23 | 0.25 | 2.25 | 0.414 |
| 31.43 | 13 | 71.14 | 956.37 | 0.25 | 2.25 | 0.414 |
| 33.77 | 14 | 71.14 | 1027.51 | 0.25 | 2.25 | 0.414 |
| 36.11 | 15 | 71.14 | 1098.65 | 0.25 | 2.25 | 0.414 |
| 38.45 | 16 | 71.14 | 1169.79 | 0.25 | 2.25 | 0.414 |

### Example 9

### Continuous Harvest of a Flat Panel Photobioreactor at Different Specific Light Levels and Algal Biomass Concentration

Nannochloropsis sp. were used to inoculate 4 feet wide by 4 feet long by 1.5 inch deep (1.2192 meters by 1.2192 meters by 0.0381 meters) flat panel photobioreactors. Algae were grown in the photobioreactor at a fixed irradiance of 39.23 mols photons/m²/day (eight 54 watt fluorescent T5 bulbs per side). When the concentration reached 4 g/L and the irradiance was increased to 78.43 mols photons/m²/day (sixteen 54 watt fluorescent T5 bulbs per side). The algae biomass concentration in these photobioreactors reached 7.45 g/L over a five day period.

The environmental parameter set points for all four treatments (as described below) were the same. The temperature was kept at 25° C. via a chiller plumbed inline to a chilling coil that ran centrally down the horizontal access of each tank. The gasses (air and CO₂) were delivered to the treatment tanks via ¼ inch inside diameter tubing connected to a ½ inch PVC line with 1 mm holes. The PVC gas delivery line was placed centrally at the bottom of the tank to allow for mixing and to reduce stratification of the culture. Air was used to apply agitation to the treatment tanks Medo LA-120 commercial air pumps with an individual capacity of 146 L/minute of air were used to deliver air to the treatment tanks One air pump provided air for two treatment tanks A rotameter was used to control the rate of air flow to the treatment tanks Each treatment received a constant stream of 45 L/minute of air. The pH was maintained at 8.0 by injecting CO₂ into the air line. A rotameter was used to control the rate of CO₂ injection into the system. A Black Stone BL 931700 pH controller was used to measure pH in each of the treatment tanks When the pH rose above 8.0, from the production of oxygen via photosynthesis and the introduction of air, CO₂ was injected into the culture at a rate of 0.5 L/minute. The algae assimilate CO₂ as a carbon source during the photosynthetic process and produce oxygen as a byproduct thus effecting pH. The nutrient solution for all the treatments was comprised of a modified f/2 media which involved the supplementation of nitrate at a concentration of 500 mg/L on a daily basis to maintain a minimum concentration of 500 mg/L nitrate in solution. The salinity for each treatment was 35 ppt.

The lighting system for the experiment was a Sun Blaze T-5 48 inch lighting fixture (121.92 cm long by 55 cm wide). Each lighting array housed a total of eight T-5 high output plant grow lights (54 Watts). Four lighting fixtures per side per treatment tank, one on each side, were placed approximately 15 centimeters from the face of the photobioreactor. All of the photobioreactor experiments were run on a 24 hour light cycle.

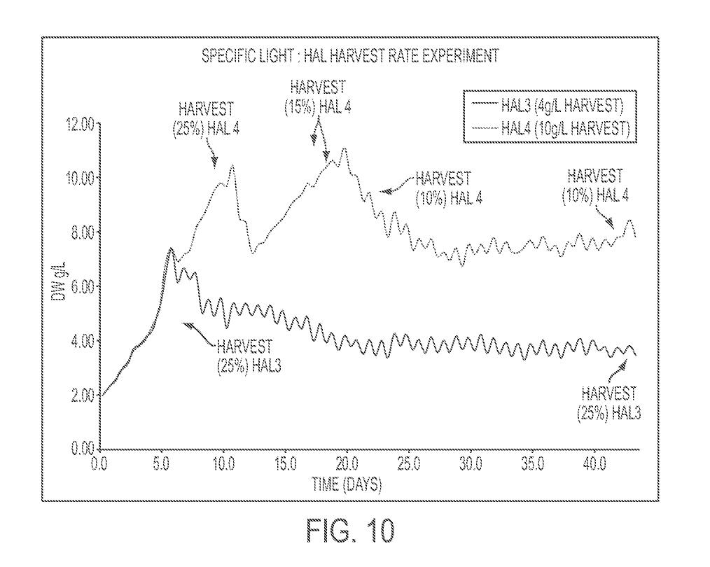

The first experimental treatment involved a daily harvest of 25% of the PBR's active volume over a 6 hour period. Keeping the irradiance level at 78.43 mol photons/m² day, the algae biomass concentration (which was 7.45 g/L prior to harvest) decreased over time reaching a steady state concentration of approximately 4.00 g/L. This continuous culture method yielded approximately 1.08-1.19 grams algae/liter day (representing the algae steady state growth rate) for 57 days as shown in FIG. 10. Once steady state was reached (at a concentration of around 4 g/L the specific light remained constant at 0.499 mol photons/g-day.

The second experimental treatment utilized a similar flat panel photobioreactor of the same dimensions was run concurrently with the first treatment to determine if a standing algae biomass concentration of 10.00 g/L at a harvest rate of 10% volume per day would generate greater yields. The environmental parameters were the same for both treatments and the nutrient solutions were maintained at the optimal levels for growth. The irradiance for this treatment was 78.43 mols photons/m²/day, which is the same as for the 4.00 gram liter⁻¹ treatment.

At a harvest rate of 10% volume per day, the standing algae biomass concentration decreased below 10.00 g/L until it reached a steady state at approximately 8.00 g/L. This reactor yielded approximately 0.80 g/L day⁻¹. This treatment continued daily harvests for 57 days. The specific light utilized in this experiment was calculated for this continuous harvest treatment to be 0.257 mol photons/g-day.

Thus, although the reactor yield was reduced by 20% (from about 1 g/L-day to 0.8 g/L-day) the specific light required to maintain this yield dropped by a significantly greater percentage of 51% (i.e. from 0.499 mol photons/g-day to 0.257 mol photons/g-day).

FIG. 10 shows the standing algae biomass concentration for the two treatments in this experiment. The algae biomass concentration in g/L is plotted over time in days. The graph indicates when and what harvest rates were applied to the culture. The 4 gram liter⁻¹ treatment with an applied specific light level of 0.499 mol photons/g-day yielded a productivity rate of about 1 gram liter⁻¹ day⁻¹. The 8-10 gram liter⁻¹ treatment at a specific light level of 0.257 mol photons/g-day, yielded a productivity of 0.8 g/L-day

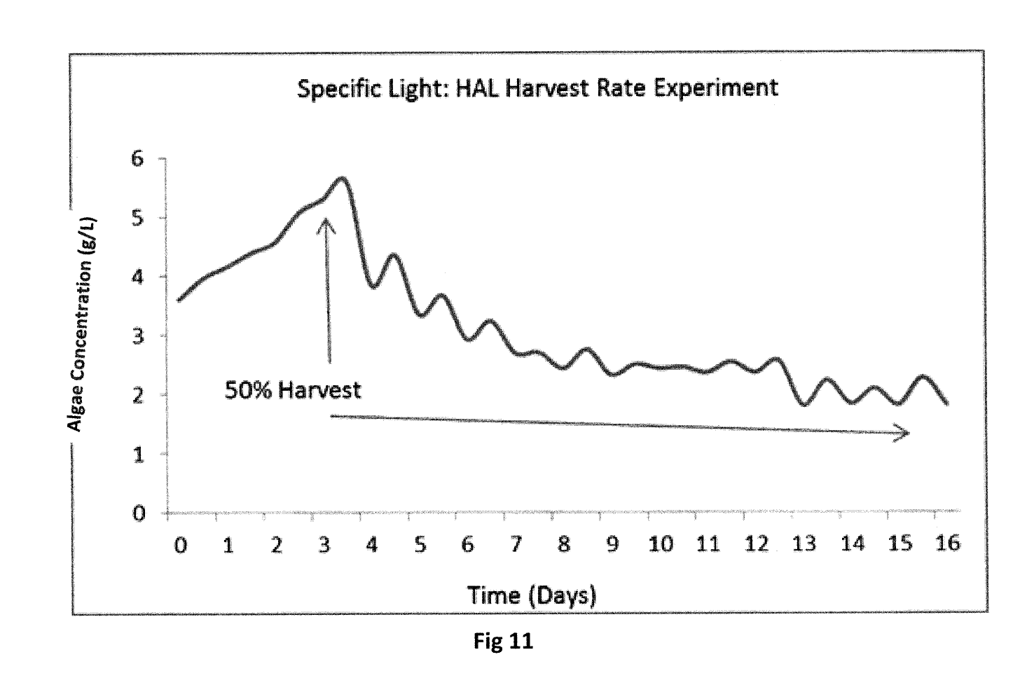

Finally, an experiment was run to demonstrate that even at different cell concentrations, similar algae production yields can be obtained provided the PBR is irradiated with the same specific light. A flat panel photobioreactor was set up to grow a standing algae biomass concentration of 2 g/L at a harvest rate of 50% volume per day. The same flat panel photobioreactor and procedures used in the previously described experiments were applied to this one. Harvest was carried out over a 6 hour period. A steady state concentration of 2 g/L at a 50% volume harvest rate per day was achieved as shown in FIG. 11. When PBRs are operating in harvest mode, approximately the same amount of algae/liquid volume removed from the PBR is simultaneously replaced with an equivalent amount of water, medium, and/or nutrients over the stated time period.

The specific light utilized in this experiment was calculated for this continuous harvest treatment to be 0.489 mol photons/g-day, and the algae yield of 1 g/L-day was obtained. This yield is similar to the first experiment which operated at a algae concentration of 4 gm/L with a similar specific light of 0.499 mol photons/g-day.

As shown in FIG. 11, the data collected in these trials demonstrates that when an optimal amount of light is applied to a photobioreactor that is growing phototrophic algae, the optimal theoretical yield of 1 gram liter⁻¹ day⁻¹ can be achieved. The optimal theoretical yield is determined to be approximately 1 gram liter⁻¹ productivity regardless of the standing biomass concentration. The optimal specific light value for maintaining growth rates in a continuous growth system was determined from the series of experiments to be approximately 0.41-0.49 mol photons/g-day. Furthermore, a specific light value of 0.4-0.5 mol photons/g-day will enable algal growth production rates of approximately 1 gram liter⁻¹ day⁻¹ in both continuous and batch cultures.

### Example 10

### The Impact of High Specific Light on Algal Growth Rates

It was shown in Example 9 that high growth rates (of about 1/L-day) can be achieved with a specific light of 0.48 to 0.49 mol photons/g-day. In example 10, the effects on algae growth rates when specific light was further increased to around 0.8 mol photons/g-day was examined.

Nannochloropsis strain 202-3 was used to inoculate a 4 feet wide by 4 feet long by 1.5 inch deep (1.2192 meters by 1.2192 meters by 0.0381 meters) flat panel photobioreactor. Algae were grown for 6 days in the photobioreactor with an irradiance of 19.35 mols photons/m²/day (eight 54 watt fluorescent T5 bulbs per side). This was calculated to equal a specific light irradiance of 0.489 mol photons/g-day. This specific light irradiance was set at the specific light irradiance level that showed the highest growth rates, as extrapolated based on the data obtained during the experiments (Example 9) described previously in the instant application.

When the algae concentration reached 4 g/L, after 5.7 days, a continuous harvest of 100% of the PBR volume over a 24 hour period was commenced. Note that when PBRs are operating in harvest mode, approximately the same volume of algae/liquid harvested from the PBR is simultaneously replaced within an equivalent volume of water, medium, and/or nutrients.

The objective of using a higher harvest rate was to reach a steady state operation with a low algae concentration in the PBR, which in turn would result in an elevated specific light value. Three samples were taken daily from the PBR harvest stream and used to determine the dry weight algae concentration in the PBR at the time of sampling.

After three days of harvesting, the standing algae biomass concentration reached 1.30 g/L-day. Harvest continued for 17.7 days before the harvest dry weight began to drop below 1.00 g/L-day. After a total culture time of 19.7 days, the algae was unable to maintain a production rate of about 1.00 g/L-day. The average harvest weight over the initial 9 day harvest period was 1.01 g/L-day. However, the harvest productivity reached 0.64 g/L-day by day 12, the end of the trial. The harvest productivity averaged approximately 0.93 g/L-day over a 12 day period. Throughout the duration of the trial the standing algae biomass density declined over time.

At day 10 the culture began to exhibit a coloration change from green to a light yellow which is indicative of oil phase when physiological stress is applied to the algae. Such stress can be induced by the removal of nitrate with the presence of another environmental parameter such as increased temperature or light. In this particular experiment, nitrate and nutrients were added to the tank on a daily basis.

Since the algae concentrations were reduced to around 1 g/L, the specific light utilized for this continuous harvest treatment was calculated to be 0.98 mol photons/g-day. Thus, the results indicate that prolonged exposure to light at this specific light level of 0.98 mol photons/g-day induced stress in the algae. This is hypothesized to be a function of light stress on the algae in which the physiological limits of the algae have been maximized and exhausted over time under 24 hour specific light exposure.

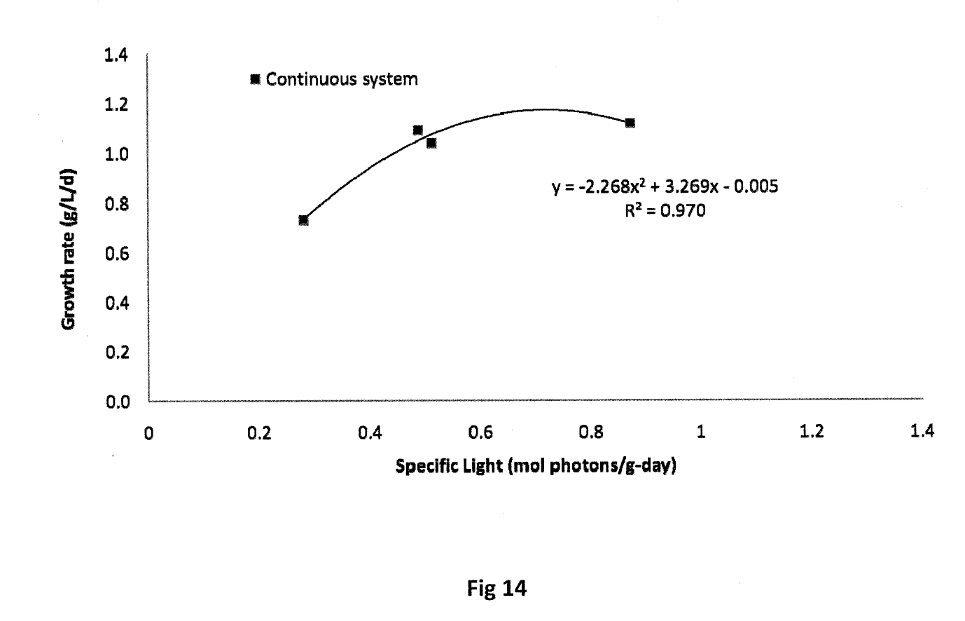

FIG. 14 shows a plot of the data obtained by the methods described in Examples 9 and 10 (continuous production).

While errors were identified in the batch production data that required estimated values to be substituted, as described previously, it is believed that it can still be utilized as a modeling tool to inform algae production development. It is believed that batch production data can be generated that would be indicative of what would actually happen if an algal mass is cultured under different specific light levels. The continuous production data was obtained in a more controlled manner, and no data was excluded from its analysis, so it is believed that this data is reliable. As the corrected batch production data closely tracks the continuous production data, it is believed that the exclusion of the outlying points was reasonable in this context.

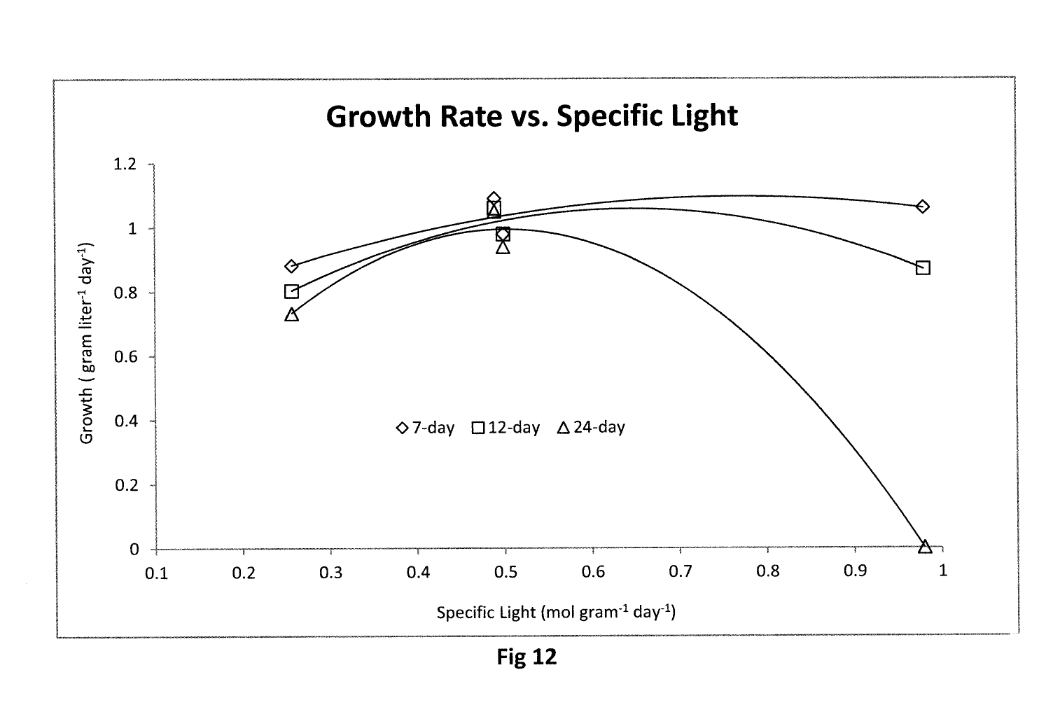

As shown in FIG. 12, while the continuous system data point at a specific light of around 0.8 mol photons/g-day did grow at a rate of about 1 g/L-day, the results of example 10 indicate that this growth rate was unsustainable over longer periods due to induced stress in the algae. FIG. 12 is a plot of the three time cycles described above and represents the application of 0.98 mols photons per gram per day over 7, 12, and 24 days. This data shows that while three culture conditions showed similar levels of growth during the lower specific light levels, as the specific light levels increased, the growth rates began to separate by day 12. By day 24, exposure to about 0.98 mols photons per gram per day exhibited signs of stress, and ceased growth. It is believe that this is due to the physiological demand put on the algae over time.

This finding was surprising as it was expected that increased growth rates and productivity would have occurred at the elevated specific light value. Clearly at these elevated specific light values it is not possible to sustain either growth or oil production over significant lengths of time.

As shown below in Example 11, while the oil production rates by day 4 were similar between the 0.6 and 0.8 mol photons/g-day specific light levels, the culture could not be sustained for as long as the culture irradiated with a specific light level of 0.6 mol photons/g-day. This means that the culture over time, when irradiated at about 0.8 mol photons/g-day is less productive than that of a culture over time irradiated at about 0.6 mol photons/g-day.

### Example 11

### Oil Production Utilizing Specific Light

Nannochloropsis mutant 202-3 algae was grown in a 2 feet by 2 feet photobioreactor for seven days under the growth conditions described in Example 9 until the algae biomass concentration reached approximately 6 g/L. In order to begin the oil production phase, the algae biomass was then split into four 2 feet by 2 feet photobioreactors and diluted to a concentration of 1 g/L.

The environmental parameter set points for all four treatments were the same. The temperature was kept at 25° C. via a chiller plumbed inline to a chilling coil that ran centrally down the horizontal access of each tank. The gasses (air and CO₂) were delivered to the treatment tanks via ¼ inch inside diameter tubing connected to a ½ inch PVC line with 1 mm holes. The PVC gas delivery line was placed centrally at the bottom of the tank to allow for mixing and to reduce stratification of the culture. Air was used to apply agitation to the treatment tanks Medo LA-120 commercial air pumps with an individual capacity of 146 liters minute⁻¹ of air were used to deliver air to the treatment tanks One air pump provided air for two treatment tanks A rotameter was used to control the rate of air flow to the treatment tanks Each treatment received a constant stream of 45 liters minute⁻¹ of air. The pH was maintained at 8.0 via CO₂ injection into the air line. A rotameter was used to control the rate of CO₂ injection into the system. A Black Stone BL 931700 pH controller was used to measure pH in each of the treatment tanks When the pH rose above 8.0, from the production of oxygen via photosynthesis and the introduction of air, CO₂ was injected into the culture at a rate of 0.5 liters minute⁻¹.

The algae assimilate CO₂ as a carbon source during the photosynthetic process and produce oxygen as a byproduct, thus effecting pH. The nutrient solution for all the treatments was comprised of a modified f/2 media. Additional nitrate was added to the standard f/2 culture media at a concentration of 500 mg/L on a daily basis to maintain a minimum concentration of 500 mg/L nitrate in solution. The salinity for each treatment was 35 ppt.

This process and operating procedure were utilized in flat panel photobioreactor cultures which maintain optimal productivity of algae relative to standing biomass concentration. The lighting system for the experiment consisted of a Sun Blaze T-5 48 inch lighting fixture (121.92 cm long by 55 cm wide). Each lighting array houses a total of eight T-5 high output plant grow lights (54 Watts). Four lighting fixtures per side per treatment tank, one on each side, were placed approximately 15 centimeters from the face of the photobioreactor. All of the photobioreactor experiments were run on a 24 hour light cycle.

Each reactor was exposed to a different specific light level: 0.2, 0.4, 0.6, and 0.8 mol photons/g-day. No nitrogen was supplemented after the split in order to induce nitrogen depletion and stress the algae such that the culture entered the oil production phase. A sample (n=3) was taken on day 0 from the growth phase bioreactor in order to establish the initial oil content of the algae before beginning the oil production phase. Samples (n=3) were taken daily from each of the four oil phase reactors to assess biomass production during the oil phase. Samples (n=3) were taken on days 2 and 4 for lipid accumulation analysis.

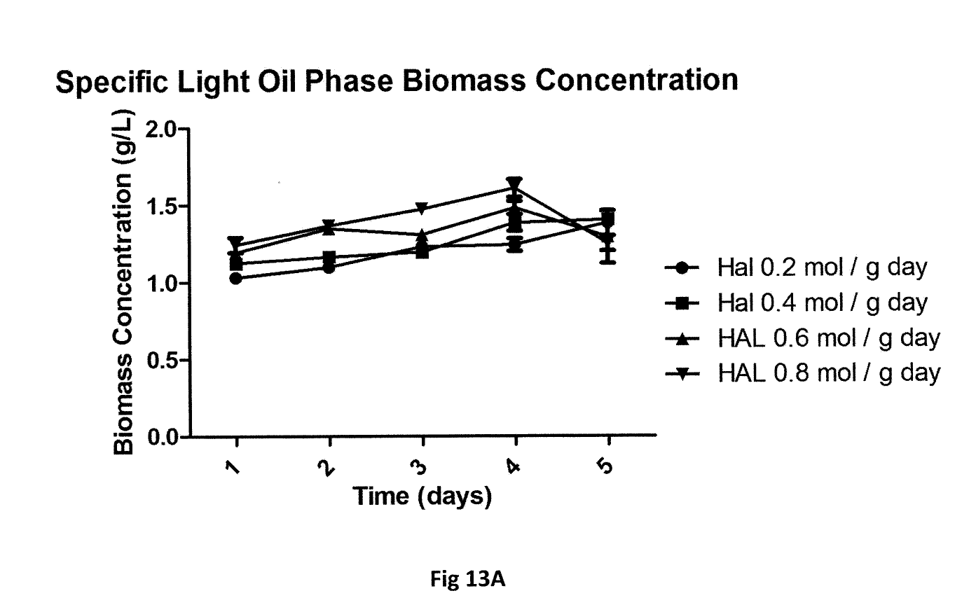

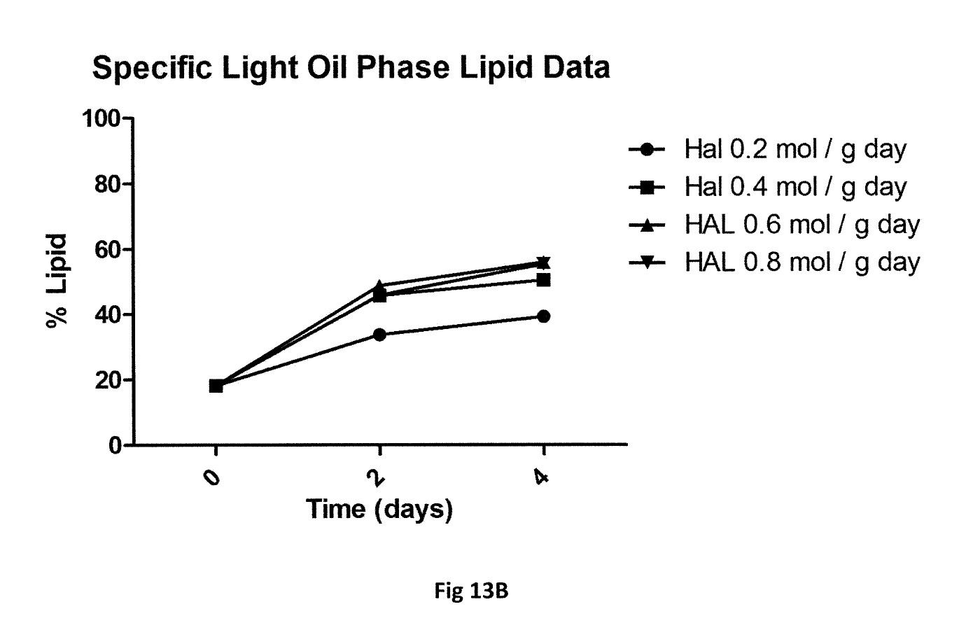

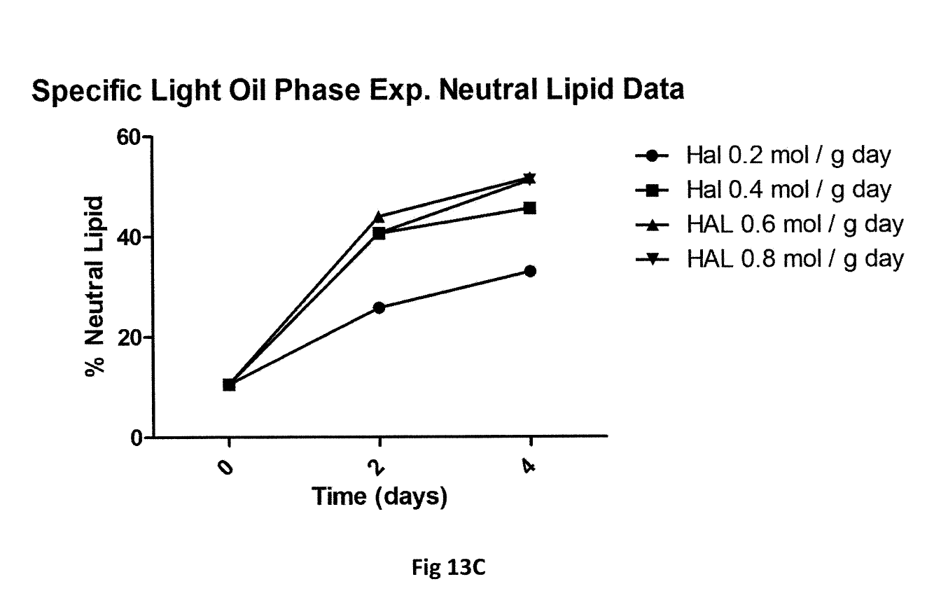

The results as shown in FIG. 13A indicated that there was no statistically significant different (p=0.1652) in the growth of the algae under the four different specific lighting conditions, as measured by the biomass concentration. This supported the prior findings that there is no statistically significant growth of the algal mass after nitrogen depletion.

The results as shown in FIG. 13A, B, and C also indicated that there was a statistically significant difference (p<0.0001) in lipid accumulation after nitrogen depletion. There was also a statistically significant difference (p<0.0001) in lipid accumulation under specific light levels of 0.2 and 0.4, 0.6 and 0.8 mol photons/g-day. There was a statistically significant difference (p<0.001) in lipid accumulation under specific light levels of 0.4 vs. 0.6 and 0.8 mol photons/g-day at day 4 and at day 2. There was a statistically significant in lipid accumulation under specific light levels of 0.4 and 0.8 mol photons/g-day at day 4, but not at day 2. There was a statistically significant change (p<0.01) in lipid accumulation under specific light levels of 0.6 and 0.8 mol photons/g-day by day 2. There was no statistically significant difference (p>0.05) in lipid accumulation under specific light levels of 0.6 and 0.8 mol photons/g-day by day 4.

As shown in FIGS. 13A, B, and C, the data suggests that Nannochloropsis mutant 202-3 can accumulate lipid at levels of specific light between 0.2-0.8 mol photons/g-day. The highest lipid content was achieved at specific light levels of 0.6 and 0.8 mol photons/g-day and their respective levels were similar by day 4. At day 2, the lipid concentration under these two light levels was statistically significantly different, but surprisingly, by day 4, the levels were very similar, approximately 51%. This implies that not only is it not necessary to operate at the highest possible level of specific light, it is actually wasteful and inefficient. While initial hypotheses contemplated operating at the specific light level that gives the maximum algal growth rate (as denoted by the extrapolated continuous process curve shown in FIG. 14), experiments such as the one described herein, show that algae oil production is actually greater at a lower specific light level.

The majority of neutral lipid accumulation occurred in the first two days of the experiment. The initial lipid content was about 10% and increase to about 40% during that time period. Specific light values of 0.4, 0.6, and 0.8 mol photons/g-day had similar neutral lipid accumulation in the first two days, however by the fourth day, the 0.6 and 0.8 mol photons/g-day had about 5% more lipid accumulation than the 0.4 mol photons/g-day treatment. This suggests that a shorter oil phase under a potentially lower specific light level (0.4-0.6 mol photons/g-day) could achieve positive results of about 40% lipids. This data can be used to model the methods, duration, and application of the oil growth phase, as shown in Example 12.

### Example 12

### Predicted Growth Data

In order to supplement the experimental data, modeling programs were used to predict culture (volumes and concentrations) and lighting conditions similar to production conditions. This type of analysis can be used to extrapolate and develop production growth plans for larger scale experiments, as well as scale up for larger scale industrial runs capable of generating sufficient amounts of algal mass and oil production for the production of biofuels as well as algal coproducts and feedstocks.

The software used to generate these models was CADSIM, a process modeling program made by Aural Systems. The solar irradiance data entered into the program was obtained from the National Renewable Energy Laboratory for the Phoenix area, and averaged from 2006-2009 to generate an average irradiance for every hour of every day of the year. The PAR sunlight was assumed to be the total solar irradiance multiplied by 45.8%, the approximate percentage of PAR in sunlight.

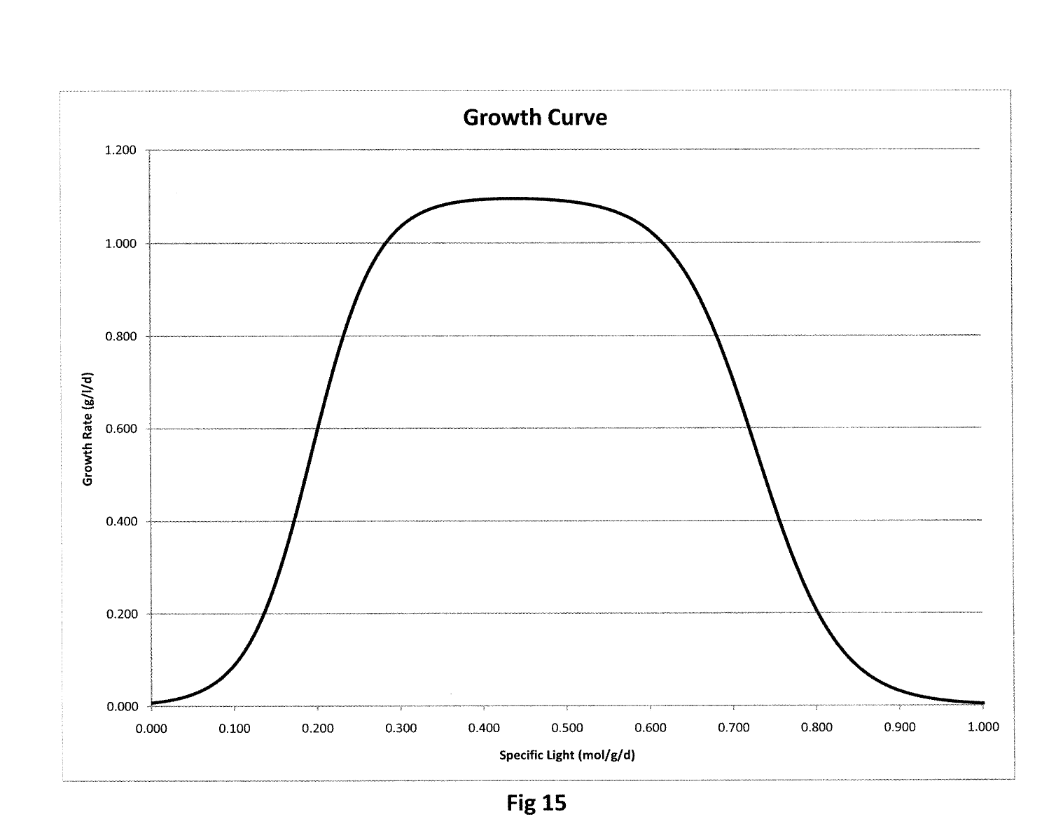

FIG. 15 depicts the predicted productivity curve when the algal growth rate is plotted against the specific light. This is the predicted version of the actual batch and continuous production data shown in FIG. 14. It is expected that if more data points were taken, the shape of the actual curves would approximate the predicted curve of FIG. 15.

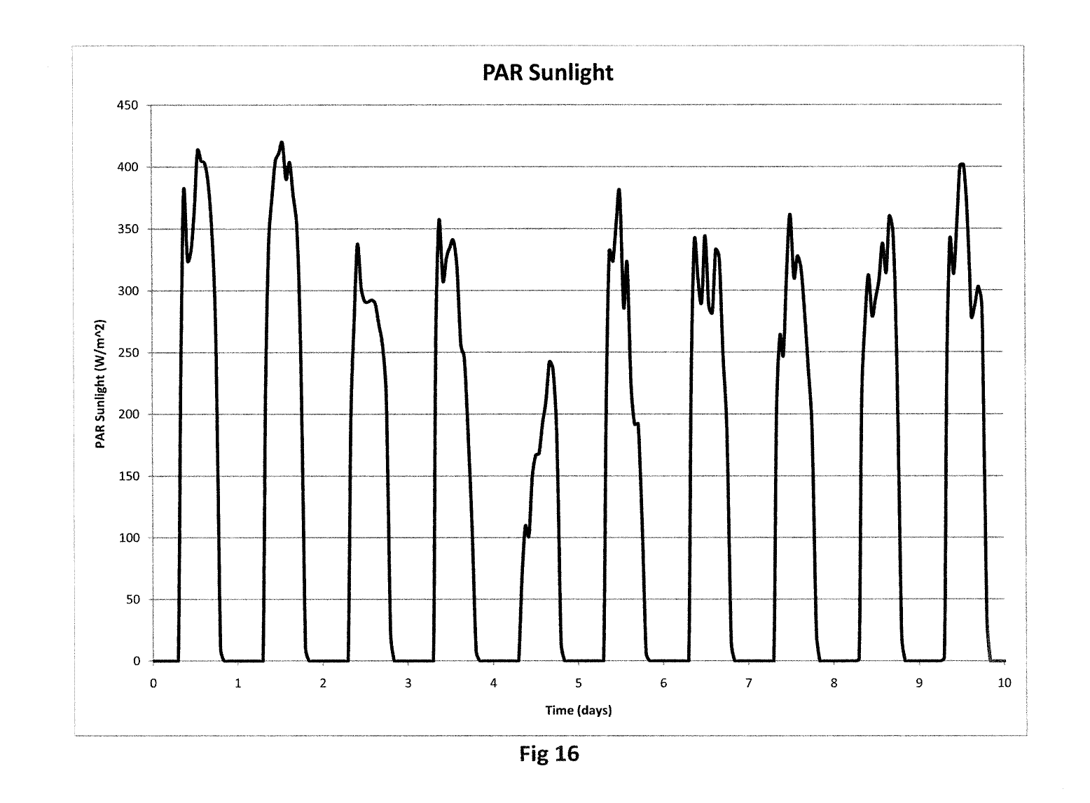

FIG. 16 shows a 10 day cycle of PAR sunlight, based on light data obtained from the National Renewable Energy Laboratory for the Phoenix area, and averaged from 2006-2009 to generate an average irradiance for every hour of every day of the year. The PAR sunlight was assumed to be the total solar irradiance multiplied by 45.8%, the approximate percentage of PAR in sunlight.

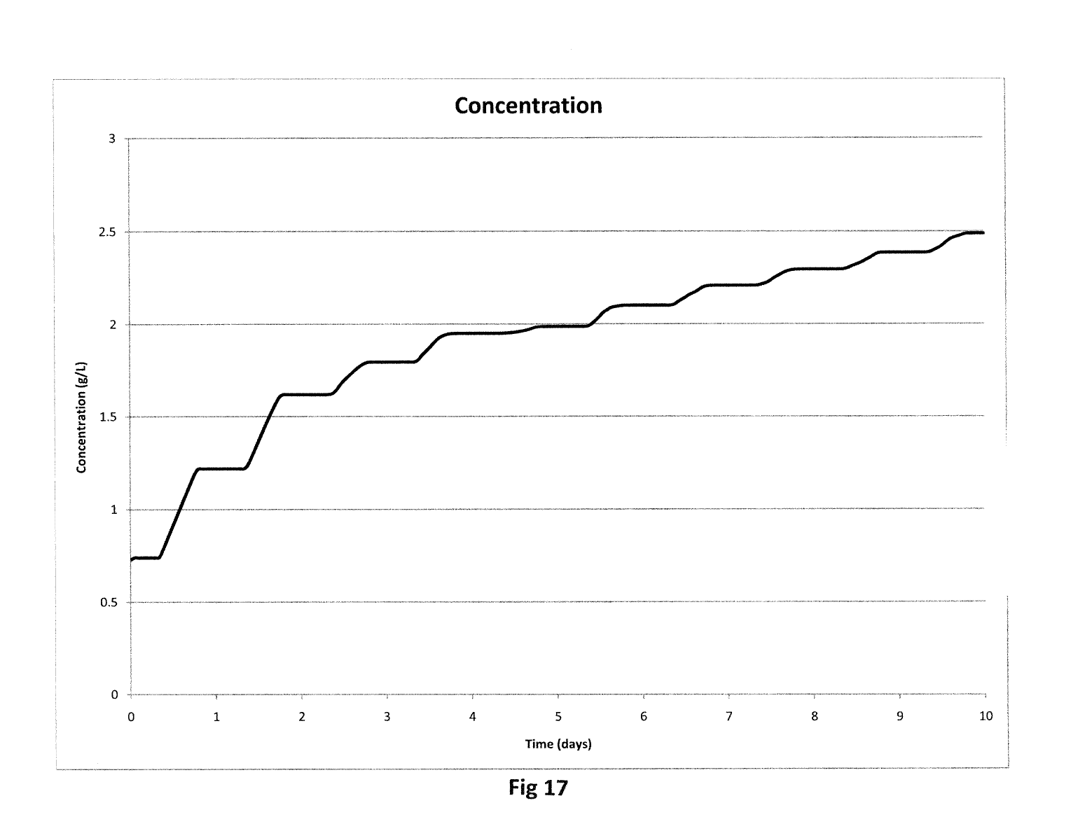

FIG. 17 shows a simulation of the concentration over time of algae growing in a photobioreactor for 10 days under PAR sunlight, at the intensities depicted in FIG. 16, without the application of specific light control.

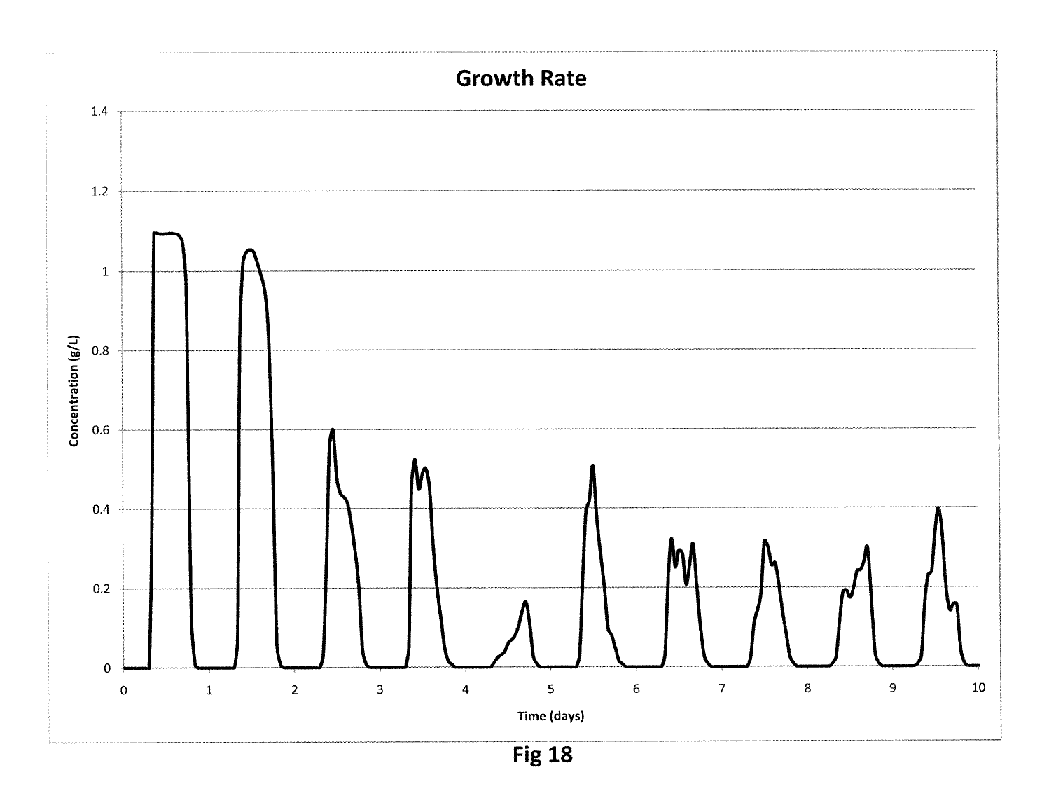

As shown in FIG. 18, under these conditions, the algae would begin growing quickly, but the growth rate would taper off over time as the algal mass increased. This is because as the algae grow, the sunlight is no longer sufficient to maintain the high growth rates seen here. The application of specific light control would allow for maintaining these growth rates, either by increasing the amount of light or diluting the algal biomass.

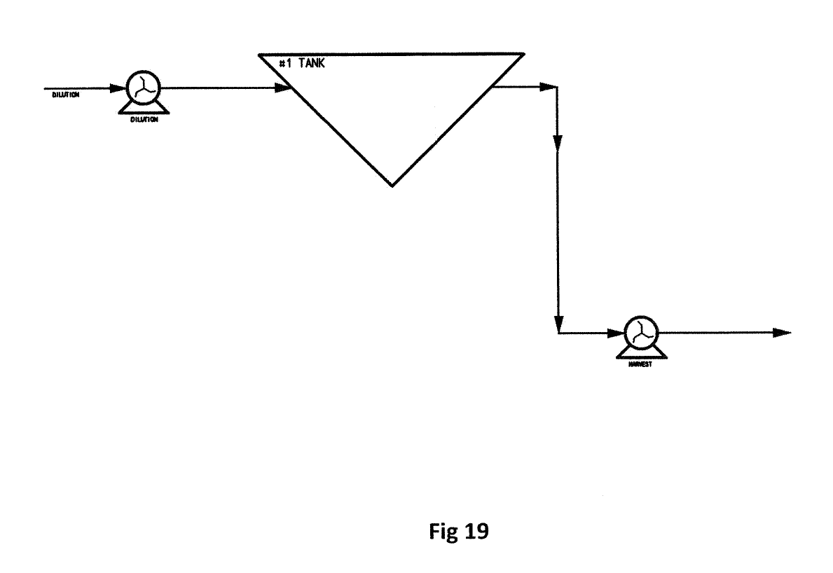

In these simulations, as PAR sunlight is used, a preferred embodiment involves the removal/dilution of algal mass in order to maintain the ratio of light irradiance to algal mass. Such an embodiment is depicted in FIG. 19 which shows a V shaped trough photobioreactor with dilution control. As the sun sets and light levels decrease, more water and/or more medium is pumped into the reactor to dilute the culture. Conversely, as the sun rises and light levels increase, less or no water and/or medium is pumped into the water in order to maintain the proper specific irradiance within a determined range. An auxiliary algal biomass can be used to increase the algal culture in response to decreasing light levels. In the alternative, in some embodiments, the PAR sunlight can be supplemented with artificial PAR light rather than diluting of the algal culture to maintain specific light levels.

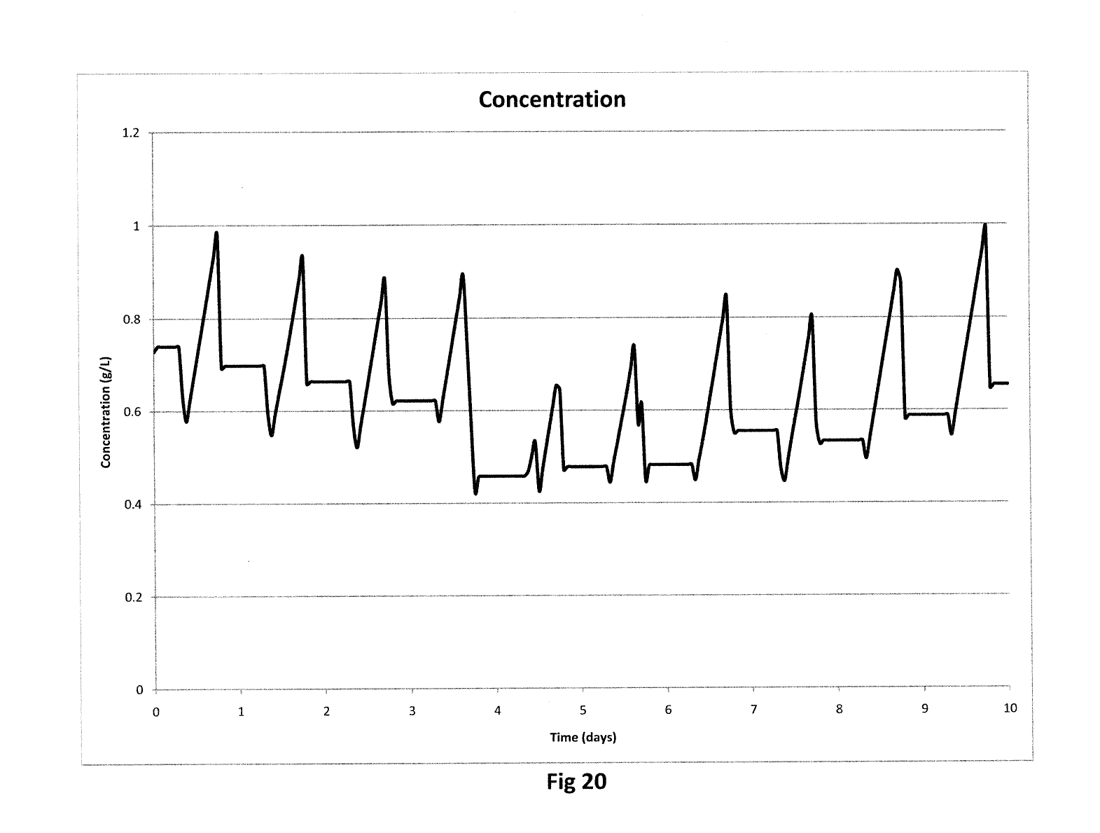

FIG. 20 is a plot of the concentration over time in a photobioreactor with specific light control. The model controls the specific light by assuming dilution and/or removal of algal biomass in the reactor in response to decreasing light levels. Here it can be observed that the periods of increasing concentration of similar high slopes, indicating high growth rates. As predicted based on the previously described experimental data, the growth rate is kept approximately constant, as demonstrated by the similarity of the slopes of the concentration of the spikes of algae growth over time, even as algal concentration increased over time.

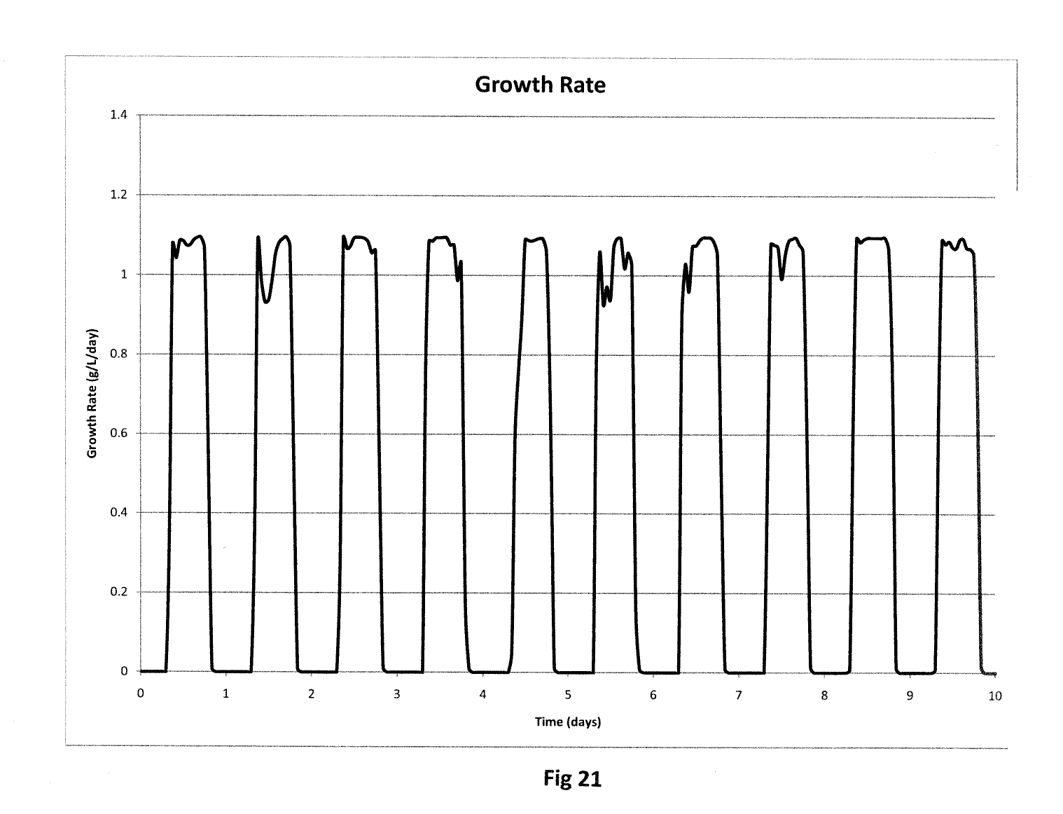

Finally, FIG. 21 shows the growth rates of such a system. This can be contrasted with the growth rate pattern seen in FIG. 18. Note that the growth rate remains constant over time, whereas in the photobioreactor without specific light control, the growth rate diminishes over time.

### Example 13

### Predicted Productivity at Maximum and Sub-Maximum Specific Light Levels

As can be seen from Table 2 below, surprisingly, the highest productivities have been calculated to occur at lower specific light values. Specific light values were selected along the specific light curves described in previous examples. These values were selected from the top of the curve, which was the point at which it was previously thought maximum production would occur, a point close to the bottom of the specific light curve, and a third point in between. This in between point, designated as the “Low Growth and Oil Rate” in Table 2, yields estimates that are significantly greater in productivity than the points above and below, the “Highest Growth and Oil Rate” and “Sub-Optimal Growth and Oil Rate,” respectively.

It is possible to predict oil productivity for a defined process configuration using the measured algae growth and oil production rates resulting from the application of known specific light quantities. The formula column of Table 2 presents the methodology used to calculate oil productivity of an algal culture. Process configurations and algal growth and oil production data were used to calculate these values. The process configuration used for these calculations is shown in FIG. 7.

Initially, the oil produced per day in grams per day and the energy required to produce a gram of oil were calculated for the defined process configuration. Productivity is scaled to the amount of oil that would be produced by the sun's energy received annually on an acre of land. The sun's energy flux (MJ/m²/year) for Phoenix, Ariz. were used for the scaling. Since about 48.5% of the sun's energy is PAR, the sun's energy flux value was adjusted for this in the calculations.

Table 2 shows that 5, 226 gallons per acre per year of oil would be produced when operating at specific light levels that produced the maximum rates of growth. The specific light level associated with the maximum rate of growth was taken from the data set shown in FIG. 14. The maximum oil production rate data used in the calculation was taken from the data set shown in FIG. 13.

Surprisingly, projections show that significantly higher quantities of oil can be produced by operating at specific light levels below that which cause maximum growth rates. The high productivity case presented in Table 2 shows that an oil productivity of 11, 832 gallons per acre per year can be attained when using measured growth and oil production rates that result when the algae are irradiated with specific light values that are lower than the values used to produce maximal growth and/or oil production rates. These data was also extrapolated from the results shown in FIGS. 13 and 14.

TABLE 2
Calculated Productivity at Different Growth and Oil Production Rates

| | | | | Highest rate case | | High productivity case | |
|---|---|---|---|---|---|---|---|
| | | | | Growth | Oil | Growth | Oil |
| | | Unit | Formula | phase | phase | phase | phase |
| Lab data Inputs | | | | | | | |
| A1 | Growth rate | g/L/d | | 1.12 | 0 | 0.8 | 0 |
| A2 | Oil conv rate | %/d | | 2% | 9% | 2% | 5% |
| A3 | Specific Irradiance | mol photons/ | | 0.875 | 0.8 | 0.257 | 0.2 |
| | | g-day | | | | | |
| Process Inputs | | | | | | | |
| B1 | Individual PBR | L | | 100 | 100 | 100 | 100 |
| | volume | | | | | | |
| B2 | Algae conc out of | g/L | | 7.32 | 1 | 7.32 | 1 |
| | growth reactor | | | | | | |
| B3 | Oil % out of process | | | 20% | 40% | 20% | 40% |
| Mass and process calcs | | | | | | | |
| C1 | Flow rate | L/d | B1 × B2/A1 | 654 | 654 | 915 | 915 |
| C2 | Total residence time | d | B2/A1 | 7 | 2 | 9 | 4 |
| C3 | Total PBR volume | L | C1 × C2 | 4,272 | 1,404 | 8,372 | 3,472 |
| C4 | # of PBRs | | C3/B1 | 43 | 15 | 84 | 35 |
| C5 | Algae produced/day | g/d | C1 × B2 | 4,784 | 654 | 6,698 | 915 |
| C6 | Oil produced/day | g/d | C5 × B3 | 957 | 261 | 1,340 | 366 |
| Energy Required | | | | | | | |
| D1 | Specific Irradiance | mol photons/ | | 0.88 | 0.80 | 0.26 | 0.20 |
| | | g-day | | | | | |
| D2 | Sp Irr | kJ/g/d * | | 199 | 182 | 58 | 45 |
| D3 | Algae in the system | g | B2 × C3 | 31,268 | 1,404 | 61,285 | 3,472 |
| D4 | Energy Req | kJ/d | D2 × D3 | 6,216,310 | 255,206 | 3,578,606 | 157,797 |
| D5 | Energy req/g algae | kJ/g algae | D4/C5 | 1,299 | 390 | 534 | 172 |
| | produced | | | | | | |
| D6 | Energy req/g oil | kJ/g oil | D4/C6 | 6,497 | 976 | 2,671 | 431 |
| | produced | | | | | | |
| Energy available | | | | | | | |
| E1 | Sun Energy | MJ/m2/yr ** | | 9135 | 9135 | 9135 | 9135 |
| E2 | % PAR | *** | | 48% | 48% | 48% | 48% |
| E3 | PAR Sun energy on | MJ/acre/yr | | 17,740,761 | 17,740,761 | 17,740,761 | 17,740,761 |
| | 1 acre of land | | | | | | |
| Yield calculations | | | | | | | |
| F1 | kg oil/acre/yr | kg/acre/yr | E3/D6 | 2,731 | 18,173 | 6,641 | 41,149 |
| F2 | gal/acre/yr | gal/acre/yr | F1 | 785 | 5,226 | 1,910 | 11,832 |

* 1 mol photon of PAR light between 400-700 nm is 227.21 kJ energy
** based on the average irradiance in Phoenix, A over the year - data from NREL website
*** Robertson, et al, “A New Dawn For Industrial Photosynthesis,” Photosynthesis Research, 2011.

1. A method comprising:
providing an algal culture contained within a photobioreactor, the photobioreactor having at least one surface through which light may pass;
exposing the at least one surface of the photobioreactor to a light source that emits photons such that at least a portion of the algal culture is exposed to a quantity of emitted photons over a given time period;
periodically estimating the algal mass of the portion of the algal culture exposed to the quantity of emitted photons over the given time period;
in response to measuring the algal mass, increasing or decreasing at least one of (i) the quantity of emitted photons to which the portion of the algal culture is exposed over the given time period or (ii) the algal mass exposed to said quantity of emitted photons over the given time period to maintain a ratio of the quantity of photons emitted over the given time period to the algal mass within a defined range.

2. The method of claim 1 wherein the algal culture is a batch algal culture or a continuous growth algal culture.

3. The method of claim 1 wherein the light source is artificial light, natural light, or a combination of artificial and natural light.

4. The method of claim 1 wherein the light source is artificial and emits photons that comprise mainly photosynthetically active radiation.

5. The method of claim 1 wherein the defined range in which the ratio of the quantity of photons emitted over the given time period to the algal mass is maintained at less than or equal to a ratio at which a growth rate of the algal culture is maximal.

6. The method of claim 1 wherein the rate at which the emitted photons are incident upon the surface of said photobioreactor is constant, and the defined range in which the ratio of the quantity of photons emitted over the given time period to the algal mass is maintained by removing at least a fraction of said algae mass as it grows over time.

7. The method of claim 1 wherein the quantity of emitted photons to which the portion of the algae culture is exposed over time is increased in proportion to an increase in the algal mass due to algal culture growth to maintain the ratio of the quantity of photons emitted over the given time period to the algal mass.

8. The method of claim 7 wherein the algal mass increase due to growth is at a rate of about 1 gram of algal mass per liter of algal culture per day.

9. The method of claim 1 further comprising applying an environmental stress to the algal culture to induce the algal culture to produce algal lipids.

10. The method of claim 9 wherein the environmental stress is nitrate depletion, increased light, increased temperature, phosphate depletion, or nutrient depletion.

11. A method comprising:
providing an algal culture contained within a first photobioreactor, the first photobioreactor having at least one surface through which light may pass;
exposing the at least one surface of the first photobioreactor to a first light source that emits photons such that at least a first portion of the algal culture is exposed to a first quantity of emitted photons over a first time period;
periodically estimating the algal mass of the first portion of the algal culture exposed to the first quantity of emitted photons over the first time period;
in response to measuring the algal mass of the first portion, increasing or decreasing at least one of (i) the first quantity of emitted photons to which the first portion of the algal culture is exposed over the first time period or (ii) the algal mass of the first portion exposed to the first quantity of emitted photons over the first time period to maintain a first ratio of the first quantity of photons emitted over the first time period to the algal mass of the first portion within a first defined range;
transferring a second portion of the algal culture contained within the first photobioreactor to a second photobioreactor, the second photobioreactor having at least one surface through which light may pass;
applying an environmental stress to the second portion of the algal culture to induce the second portion of the algal culture to produce algal lipids;
exposing the at least one surface of the second photobioreactor to a second light source that emits photons such that at least a third portion of the algal culture is exposed to a second quantity of emitted photons over a second time period;
periodically estimating the algal mass of the third portion of the algal culture exposed to the second quantity of emitted photons over the second time period;
in response to measuring the algal mass of the third portion, increasing or decreasing at least one of (i) the second quantity of emitted photons to which the third portion of the algal culture is exposed over the second time period or (ii) the algal mass of the third portion exposed to said second quantity of emitted photons over the second time period to maintain a ratio of the second quantity of photons emitted over the second time period to the algal mass of the third portion within a second defined range.

12. The method of claim 11 wherein at least one of the first and the second photobioreactor is a batch photobioreactor or a continuous growth photobioreactor.

13. The method of claim 11 wherein at least one of the first and the second light source is artificial light, natural light, or a combination of artificial and natural light.

14. The method of claim 11 wherein at least one of the first and the second light sources is artificial and emits photons that comprise mainly photosynthetically active radiation.

15. The method of claim 11 wherein the first defined range in which the ratio of the first quantity of photons emitted over the first time period to the algal mass of the first portion is maintained less than or equal to a ratio at which a growth rate of the algal culture is sustainable.

16. The method of claim 11 wherein the rate at which the emitted photons are incident upon the surface of the first photobioreactor is constant, and the first defined range in which the ratio of the first quantity of photons emitted over the first time period to the algal mass of the first portion is maintained by removing at least a portion of said algae mass of the first portion as it grows over time.

17. The method of claim 11 wherein the first quantity of emitted photons to which the first portion of the algae culture is exposed over time is increased in proportion to an increase in the algal mass of the first portion due to algal culture growth to maintain the ratio of the first quantity of photons emitted over the first time period to the algal mass.

18. The method of claim 11 wherein the first light source and the second light source are different.

19. The method of claim 11 wherein the environmental stress is nitrate depletion, increased light, increased temperature, phosphate depletion, or nutrient depletion.

20. The method of claim 11 wherein the first defined range differs from the second defined range.
